(19) United States
(12) Patent Application Publication
(10) Pub. No.: US 2024/0209016 A1
(43) Pub. Date: Jun. 27, 2024

(54) FLUORESCENT NUCLEOSIDE PHOSPHATES

(71) Applicant: STEALTH LABELS BIOTECH AB, Gothenburg, Västra Götaland County (SE)

(72) Inventors: Elin ESBJÖRNER WINTERS, Gothenburg (SE); Marcus WILHELMSSON, Gothenburg (SE); Pauline PFEIFFER, Gothenburg (SE); Audrey GALLUD, Gothenburg (SE); Jesper NILSSON, Gothenburg (SE)

(21) Appl. No.: 18/555,180

(22) PCT Filed: Apr. 11, 2022
(86) PCT No.: PCT/EP2022/059666

§ 371 Date: Oct. 12, 2023

(30) Foreign Application Priority Data
Apr. 12, 2021 (GB) 2105194.1

(51) Int. Cl. C07H 19/14; C12N 15/10

(52) U.S. Cl. CPC C07H 19/14; C12N 15/10

(57) ABSTRACT

This specification discloses novel phosphate compounds of formula (I) useful for labelling RNA in-cellulo. The phosphates are spontaneously taken up by cells and may be used as substrates for RNA synthesis once across the cell membrane. This technology therefore offers a general route to understanding the biological behaviour of RNA of interest, including RNA based drugs.

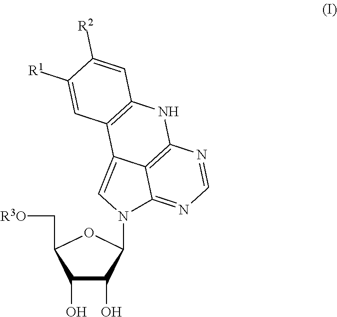

### FIELD

This specification relates to modified fluorescent nucleoside phosphates and their use to elucidate biological mechanisms.

### BACKGROUND

RNA plays a fundamental role in biology. It is the main player of the central dogma of biochemistry and a crucial regulator of gene expression via for instance micro and small interfering RNA, as well as through its intrinsic catalytic activity. It has, for these reasons, also emerged as a highly promising and versatile new drug modality: since RNA therapeutics have the potential to modify cellular function at the translational level, they may open up new opportunities to address previously undruggable targets.

An increased molecular and mechanistic knowledge of the biological processes involving RNA is therefore vital to understanding diseases and treat them. For example, there is a growing body of evidence suggesting that the key to unleashing the full potential of RNA-based drugs lies in understanding the processes of cell uptake and endosomal release (Dowdy, S. F., Nat. Biotechnol. 35, 222-229, [2017]). Regardless of the endocytosis mechanism, the delivery of a nucleic acid cargo to the cytoplasm always relies on endosomal escape, the understanding of which, despite extensive investigations, remains elusive (Crooke, S. T. et al., Nat. Biotechnol. 35, 230 [2017]; Pei, D. & Buyanova, M., Bioconjugate Chem. [2018]). In this context, tracking of endogenous and exogenous (therapeutic) RNAs inside cells, including their translocation, localization, splicing and degradation, is of great importance.

Recent advances have resulted in the development of a broad spectrum of tools and probes by which RNA can be analysed and quantified, but they generally involve heavily modified oligonucleotides with properties significantly different from natural ones, potentially resulting in loss of ability to be recognized and processed by the enzymatic machinery of cells. For example, a drawback of existing fluorescence-based technologies for studying cellular localization of RNA is that they primarily rely on highly amphiphilic and/or bulky external fluorescent constructs which could impair motility and perturb localization of the RNA and its molecular interactions with (for example) membrane constituents. In addition, a majority of these technologies are incompatible with live cell imaging (Li, Y., Ke, K. & Spitale, R. C., Biochemistry 58, 379-386 [2019]).

To overcome these issues and provide an improved method of investigating RNA mediated mechanisms this specification discloses fluorescent nucleoside phosphates that are non-cytotoxic and therefore amenable to intracellular use. The phosphates spontaneously accumulate in cultured human cells following uptake via an energy-dependent pathway, in different cell localisations depending on the molecular structure of the nucleobase (some phosphates for example amassing preferentially in the nucleus, and some in the cytosol). This allows control of downstream cell endogenous labelling processes.

To the best of the inventors' knowledge, this combination of properties has never been observed before for any other fluorescent nucleotides.

Once in the cell, enzymes accept the synthetic phosphates as canonical substrates, eventually incorporating them into endogenous cellular RNA without the need for external transfection techniques. Once incorporated, the labelled residue is minimally perturbing, allowing mechanistic study while minimising the effects of the label on the processes being observed. Because it is possible to subsequently isolate RNA from treated cells labelled in such a manner, the technology described herein offers unique opportunities for fluorescence labelling of RNA “in-cellulo” (i.e. within a cell, including in living cells).

Therefore, in summary, this specification provides a non-invasive, non-genetic way to fluorescently label endogenous RNA. It can be used to visualise—in living cells—biochemical reactions that involve RNA production, transport, processing, secretion and protein interactions. This opens the door for new ways to study and develop novel nucleic acid-based therapies.

### SUMMARY

A primary objective of the present specification is to provide modified nucleoside phosphates that can be used to conveniently and endogenously prepare fluorescently labelled RNA in-cellulo.

Accordingly, this specification describes, in part, a compound of formula (I), a physiologically cleavable precursor or a salt thereof as claimed in claim 1.

This specification also describes, in part, a process for preparing a compound of formula (I), a physiologically cleavable precursor or a salt thereof as claimed in claim 9.

This specification also describes, in part, a composition for preparing a labelled RNA molecule as claimed in claim 18.

This specification also describes, in part, the use of a compound of formula (I), a physiologically cleavable precursor or a salt thereof to prepare a labelled RNA molecule as claimed in claim 19.

This specification also describes, in part, a process for preparing a labelled RNA molecule in-vitro as claimed in claim 24.

This specification also describes, in part, a process for preparing a labelled RNA molecule in-cellulo as claimed in claim 25.

### ILLUSTRATIVE EMBODIMENTS

The invention detailed in this specification should not be interpreted as being limited to any of the recited embodiments or examples. Other embodiments will be readily apparent to a reader skilled in the art.

### General Definitions

“A” or “an” mean “at least one”. In any embodiment where “a” or “an” are used to denote a given material or element, “a” or “an” may mean one. In any embodiment where “a” or “an” are used to denote a given material or element, “a” or “an” may mean 1, 2, 3, 4, 5, 6, 7, 8, 9, 10, 100, 1000, 10000, 100000 or 1000000 (1 million).

When an embodiment includes “a” or “an” feature X, subsequent referrals to “the” feature X do not imply only one of the feature is present. Instead the above interpretation of “a” or “an” continues to apply so that “the” also means “at least one”. In other words, embodiments comprising “a feature X, where the feature X is . . . ” should be construed as “at least one feature X, where the at least one feature X is . . . ”.

“Comprising” means that a given embodiment may contain other features. For example, in any embodiment where a material “comprising” certain materials or elements is mentioned, the given material may be formed of at least 10% w/w, at least 20% w/w, at least 30% w/w, or at least 40% w/w of the materials or elements (or combination of materials or elements).

In any embodiment where “comprising” is mentioned, “comprising” may also mean “consisting of” (or “consists of”) or “consisting essentially of” (or “consists essentially of”).

With respect to embodiments of a material, “consisting of” or “consists of” means the material or element is formed entirely of the material or element (or combination of materials or elements). In any embodiment where “consisting of” or “consists of” is mentioned the given material or element may be formed of 100% w/w of the material or element.

With respect to embodiments of a material, “consisting essentially of” or “consists essentially of” means that a given material or element consists almost entirely of that material or element (or combination of materials or elements). In any embodiment where “consisting essentially of” or “consists essentially of” is mentioned the given material or element may be formed of at least 50% w/w, at least 60% w/w, at least 70% w/w, at least 80% w/w, at least 90% w/w, at least 95% w/w or at least 99% w/w of the material or element.

In any embodiment where “is” or “may be” is used to define a material or element, “is” or “may be” may mean the material or element “consists of” or “consists essentially of” the material or element.

When it is mentioned that “in some embodiments . . . ” a certain element may be present, the element may be present in a suitable embodiment in any part of the specification, not just a suitable embodiment in the same section or textual region of the specification.

When a feature is “selected from” a list, the feature is selected from a list consisting of the specified alternatives (i.e. a list of the alternatives specified and no others).

Claims are embodiments.

Modified Nucleoside Monophosphates, Diphosphates and Triphosphates

In one embodiment there is provided a compound of formula (I), a physiologically cleavable precursor or a salt thereof:

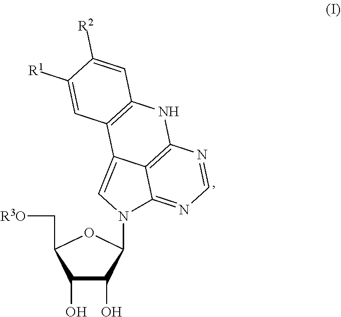

Where R¹ is selected from hydro and R² is selected from cyano, or R¹ and R² together with the atoms to which they are attached form a 6-membered carboaromatic ring and R³ is selected from —P(O)(OH)₂, —P(O)(OH)—O—P(O)(OH)₂, and —P(O)(OH)—O—P(O)(OH)—O—P(O)(OH)₂.

A “hydro” group is equivalent to a hydrogen atom. Atoms with a hydro group attached to them may be regarded as unsubstituted.

Where it is mentioned that “R¹ and R² together with the atoms to which they are attached form a 6-membered carboaromatic ring”, this may mean a phenyl ring fused to the tetracyclic heteroaromatic system in the following manner:

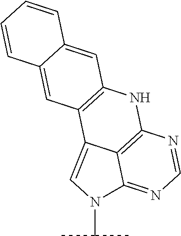

Compounds of formula (I) where R³ is —P(O)(OH)₂ have the following structure:

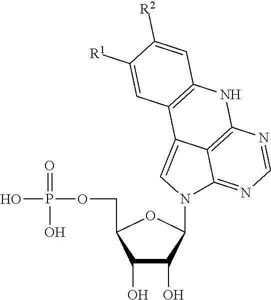

Compounds of formula (I) where R³ is —P(O)(OH)—O—P(O)(OH)₂ have the following structure:

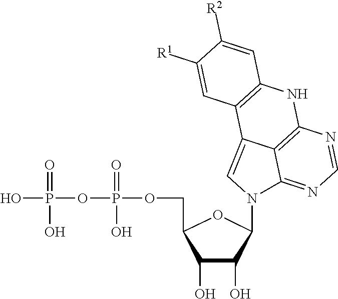

Compounds of formula (I) where R³ is —P(O)(OH)—O—P(O)(OH)—O—P(O)(OH)₂ have the following structure:

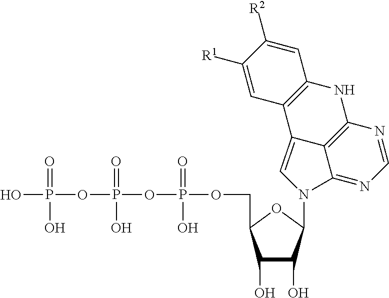

In one embodiment there is provided a compound of formula (I), a physiologically cleavable precursor or a salt thereof:

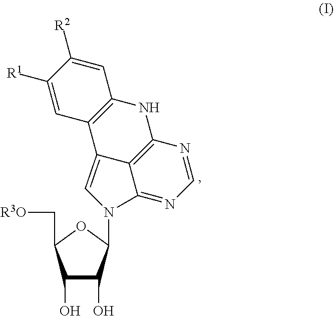

Where R¹ is hydro and R² is cyano, or R¹ and R² together with the atoms to which they are attached form a 6-membered carboaromatic ring and R³ is selected from —P(O)(OH)₂, —P(O)(OH)—O—P(O)(OH)₂, and —P(O)(OH)—O—P(O)(OH)—O—P(O)(OH)₂.

In one embodiment there is provided a compound of formula (II), a physiologically cleavable precursor or a salt thereof:

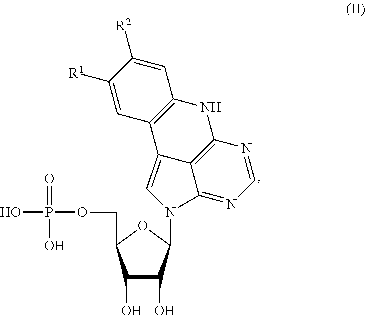

Where R¹ is selected from hydro and R² is selected from cyano, or R¹ and R² together with the atoms to which they are attached form a 6-membered carboaromatic ring.

In one embodiment there is provided a compound of formula (III), a physiologically cleavable precursor or a salt thereof:

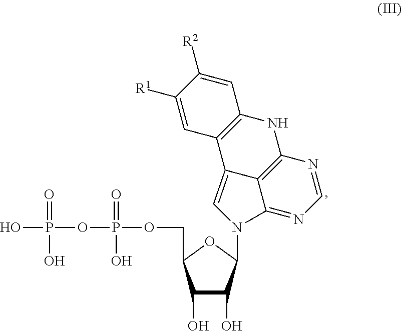

Where R¹ is selected from hydro and R² is selected from cyano, or R¹ and R² together with the atoms to which they are attached form a 6-membered carboaromatic ring.

In one embodiment there is provided a compound of formula (IV), a physiologically cleavable precursor or a salt thereof:

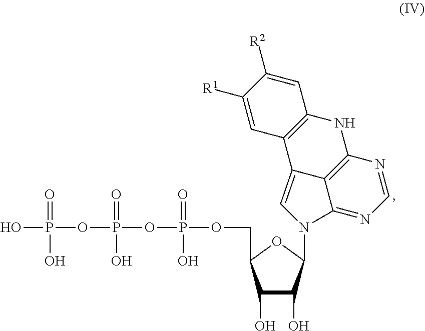

Where R¹ is selected from hydro and R² is selected from cyano, or R¹ and R² together with the atoms to which they are attached form a 6-membered carboaromatic ring.

In one embodiment there is provided a compound of formula (IV) or a salt thereof:

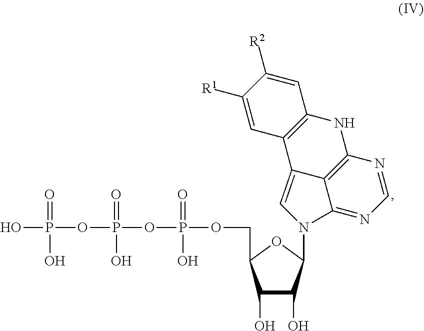

Where R¹ is selected from hydro and R² is selected from cyano, or R¹ and R² together with the atoms to which they are attached form a 6-membered carboaromatic ring.

In any embodiment where it is mentioned that a compound of formula (I) may have certain features or characteristics, those features or characteristics may also apply to a compound of formula (II), (III) or (IV).

In some embodiments R¹ is hydro and R² is cyano.

In some embodiments R¹ and R² together with the atoms to which they are attached form a 6-membered carboaromatic ring.

In some embodiments R³ is selected from —P(O)(OH)₂ and —P(O)(OH)—O—P(O)(OH)₂.

In some embodiments R³ is selected from —P(O)(OH)₂ and —P(O)(OH)—O—P(O)(OH)—O—P(O)(OH)₂.

In some embodiments R³ is selected from —P(O)(OH)—O—P(O)(OH)₂ and —P(O)(OH)—O—P(O)(OH)—O—P(O)(OH)₂.

In some embodiments R³ is —P(O)(OH)₂.

In some embodiments R³ is —P(O)(OH)—O—P(O)(OH)₂.

In some embodiments R³ is —P(O)(OH)—O—P(O)(OH)—O—P(O)(OH)₂.

In one embodiment there is provided a compound of formula (I) selected from ((2R,3S,4R,5R)-5-(8-cyano-2,3,5,6-tetraazaaceanthrylen-2(6H)-yl)-3,4-dihydroxytetrahydrofuran-2-yl)methyl dihydrogen phosphate 7a (compound 7a, 2CNqAMP), a physiologically cleavable precursor or a salt thereof, ((2R,3S,4R,5R)-5-(8-cyano-2,3,5,6-tetraazaaceanthrylen-2(6H)-yl)-3,4-dihydroxytetrahydrofuran-2-yl)methyl hydrogen triphosphate (compound 7, 2CNqATP), a physiologically cleavable precursor or a salt thereof and ((2R,3S,4R,5R)-5-(2,3,5,6-tetraazacyclopenta[de]tetracen-2(6H)-yl)-3,4-dihydroxytetrahydrofuran-2-yl)methyl hydrogen triphosphate (compound 17, pATP), a physiologically cleavable precursor or a salt thereof.

“2CNAqMP” refers to the monophosphate described as compound 7a. “2CNAqDP” refers to the diphosphate analogue of compound 7a (i.e. the analogous compound of formula (I) where R³ is —P(O)(OH)—O—P(O)(OH)₂). “2CNAqTP” refers to the triphosphate described as compound 7. Labelled residues derived from the incorporation of these compounds into RNA are “2CNqA labelled”.

“pATP” refers to the triphosphate described as compound 17. “pAMP” refers to the monophosphate analogue of compound 17 (i.e. the analogous compound of formula (I) where R³ is —P(O)(OH)₂). “pADP” refers to the diphosphate of compound 17 (i.e. the analogous compound of formula (I) where R³ is —P(O)(OH)—O—P(O)(OH)₂). Labelled residues derived from the incorporation of these compounds into RNA are “pA labelled”.

In one embodiment there is provided a compound of formula (I) selected from ((2R,3S,4R,5R)-5-(8-cyano-2,3,5,6-tetraazaaceanthrylen-2(6H)-yl)-3,4-dihydroxytetrahydrofuran-2-yl)methyl hydrogen triphosphate (compound 7, 2CNqATP) or a salt thereof and ((2R,3S,4R,5R)-5-(2,3,5,6-tetraazacyclopenta[de]tetracen-2(6H)-yl)-3,4-dihydroxytetrahydrofuran-2-yl)methyl hydrogen triphosphate (compound 17, pATP) or a salt thereof.

In one embodiment there is provided a compound of formula (I) which is ((2R,3S,4R,5R)-5-(8-cyano-2,3,5,6-tetraazaaceanthrylen-2(6H)-yl)-3,4-dihydroxytetrahydrofuran-2-yl)methyl hydrogen triphosphate or a salt thereof.

In one embodiment there is provided a compound of formula (I) or a salt thereof which is ((2R,3S,4R,5R)-5-(2,3,5,6-tetraazacyclopenta[de]tetracen-2(6H)-yl)-3,4-dihydroxytetrahydrofuran-2-yl)methyl hydrogen triphosphate or a salt thereof.

In one embodiment there is provided a compound of formula (I) which is ((2R,3S,4R,5R)-5-(8-cyano-2,3,5,6-tetraazaaceanthrylen-2(6H)-yl)-3,4-dihydroxytetrahydrofuran-2-yl)methyl hydrogen triphosphate.

In one embodiment there is provided a compound of formula (I) which is ((2R,3S,4R,5R)-5-(2,3,5,6-tetraazacyclopenta[de]tetracen-2(6H)-yl)-3,4-dihydroxytetrahydrofuran-2-yl)methyl hydrogen triphosphate.

In one embodiment there is provided a compound of formula (I) which is:

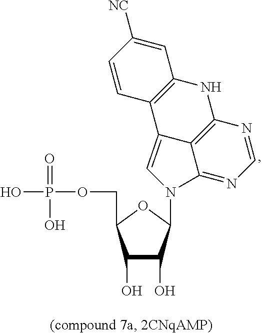

Or a physiologically cleavable precursor or salt thereof.

In one embodiment there is provided a compound of formula (I) which is:

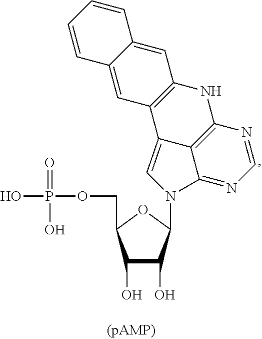

Or a physiologically cleavable precursor or salt thereof.

In one embodiment there is provided a physiologically cleavable precursor of a compound of formula (I) which is:

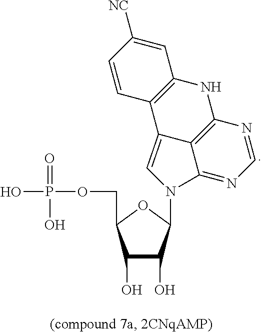

In one embodiment there is provided a physiologically cleavable precursor of a compound of formula (I) which is:

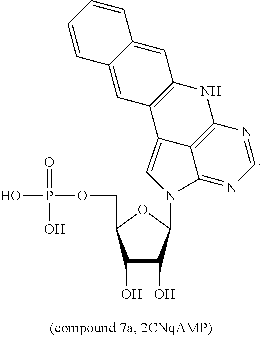

In one embodiment there is provided a salt of a compound of formula (I) which is:

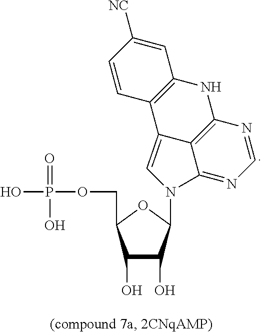

In one embodiment there is provided a salt of a compound of formula (I) which is:

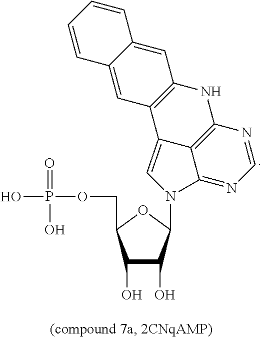

In one embodiment there is provided a compound of formula (I) which is:

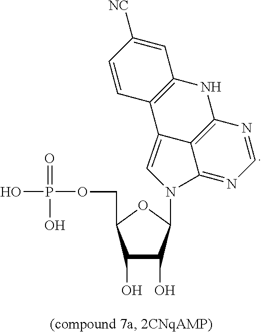

In one embodiment there is provided a compound of formula (I) which is:

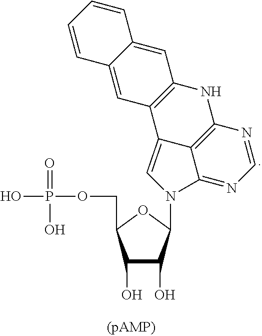

In one embodiment there is provided a compound of formula (I) which is:

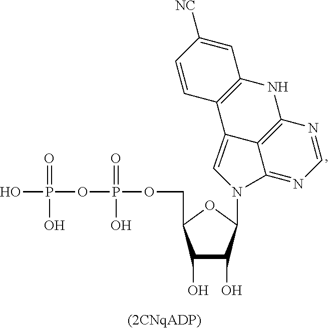

Or a physiologically cleavable precursor or salt thereof.

In one embodiment there is provided a compound of formula (I) which is:

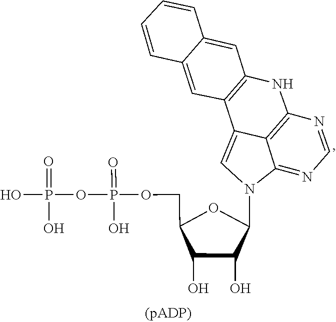

Or a physiologically cleavable precursor or salt thereof.

In one embodiment there is provided a physiologically cleavable precursor of a compound of formula (I) which is:

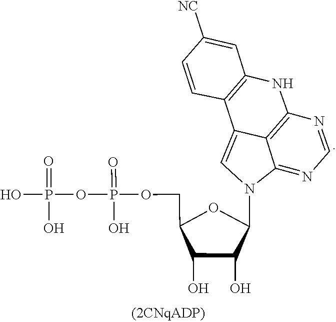

In one embodiment there is provided a physiologically cleavable precursor of a compound of formula (I) which is:

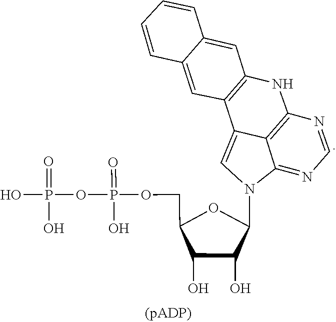

In one embodiment there is provided a salt of a compound of formula (I) which is:

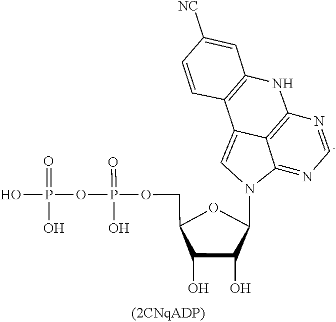

In one embodiment there is provided a salt of a compound of formula (I) which is:

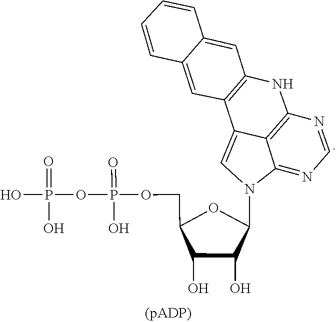

In one embodiment there is provided a compound of formula (I) which is:

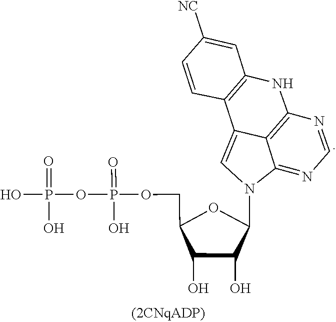

In one embodiment there is provided a compound of formula (I) which is:

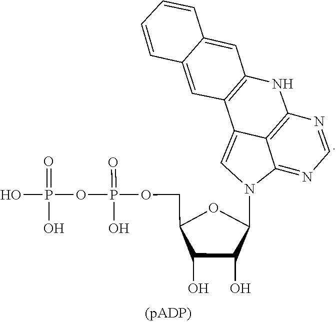

In one embodiment there is provided a compound of formula (I) which is:

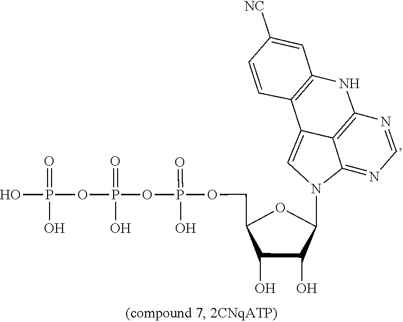

Or a physiologically cleavable precursor or salt thereof.

In one embodiment there is provided a compound of formula (I) which is:

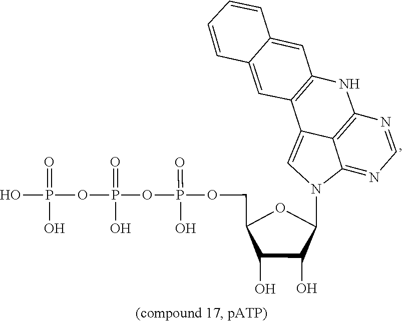

Or a physiologically cleavable precursor or salt thereof.

In one embodiment there is provided a compound of formula (I) which is:

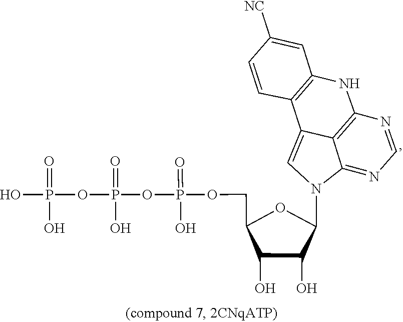

Or a salt thereof.

In one embodiment there is provided a compound of formula (I) which is:

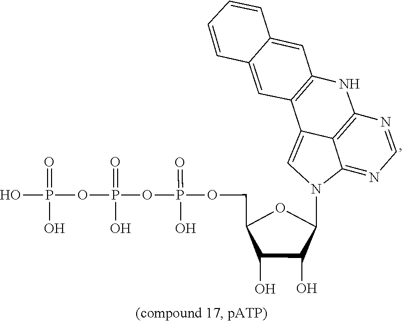

Or a salt thereof.

In one embodiment there is provided a physiologically cleavable precursor of a compound of formula (I) which is:

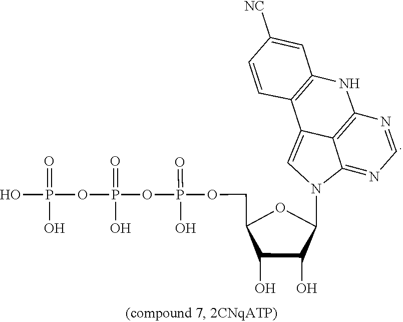

In one embodiment there is provided a physiologically cleavable precursor of a compound of formula (I) which is:

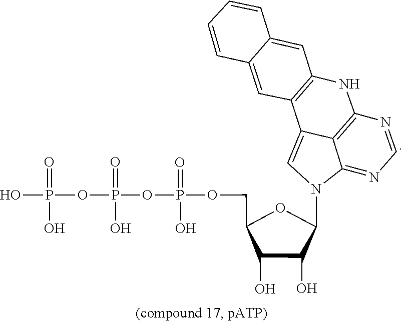

In one embodiment there is provided a salt of a compound of formula (I) which is:

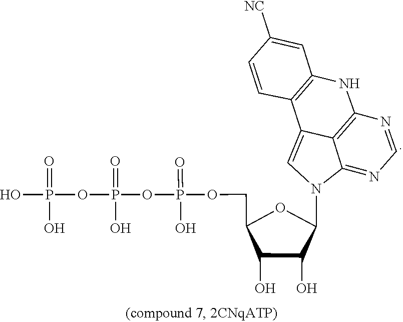

In one embodiment there is provided a salt of a compound of formula (I) which is:

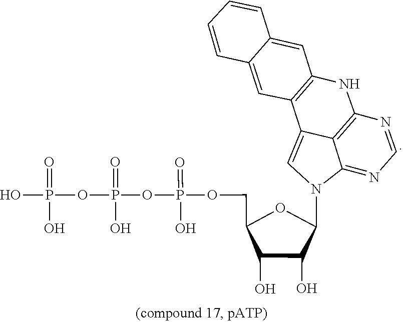

In one embodiment there is provided a compound of formula (I) which is:

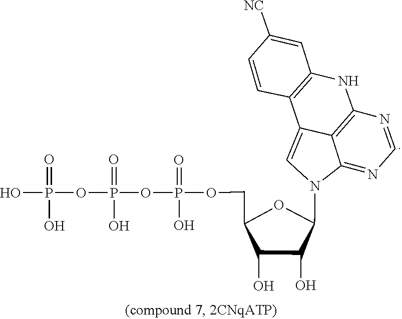

In one embodiment there is provided a compound of formula (I) which is:

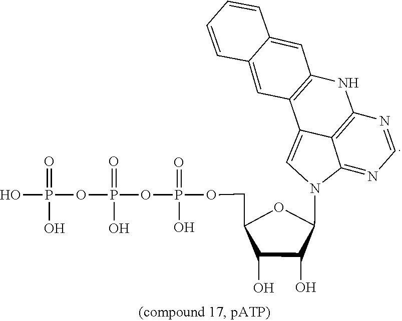

Compounds and salts described in this specification may exist as a mixture of tautomers (structural isomers resulting from the migration of a hydrogen atom that exist in equilibrium). Relevant embodiments include all tautomers of compounds of formula (I) or salts thereof.

Atoms of the compounds and salts described in this specification may exist as their isotopes. Embodiments include all compounds of formula (I) where an atom is replaced by one or more of its isotopes (for example a compound of formula (I) where one or more carbon atom is an ¹¹C or ¹³C carbon isotope, or where one or more hydrogen atom is a ²H or ³H isotope).

A physiologically cleavable precursor of a compound of formula (I) is for example one in which the mono, di- or tri-phosphate group attached to the nucleoside portion of the molecule is masked with a suitable protecting group (for example a group bound to a phosphate group oxygen atom or phosphorus atom) that may be removed under physiological conditions. When used in processes such as those described in the present specification (for example when provided to a cell) physiologically cleavable precursors of a compound of formula (I) are converted to compounds of formula (I) (e.g. by metabolism), which can then take part in cellular processes (such as cellular localisation and RNA synthesis).

In some embodiments a physiologically cleavable precursor is of a compound of formula (I)

where R³ is —P(O)(OH)₂.

In some embodiments a physiologically cleavable precursor is of a compound of formula (I) where R³ is —P(O)(OH)—O—P(O)(OH)₂.

In some embodiments a physiologically cleavable precursor is of a compound of formula (I) where R³ is —P(O)(OH)—O—P(O)(OH)—P(O)(OH)₂.

A suitable physiologically cleavable precursor of a compound of formula (I) is for example any of the groups used to prepare nucleoside phosphate and/or phosphonate prodrugs in Pradere, U. et al., Chem. Rev. 2014, 114, 18, 9154-9218 (for example in FIG. 3); Wiemer, A. J. et al., Top. Curr. Chem. 2015 (for example in Table 1); 360:115-160 and/or Wiemer, A. J.; ACS Pharmacol. Transl. Sci. 2020, 3, 4, 613-626 (for example in FIG. 2). The contents of these references are hereby incorporated by reference.

A suitable salt of a compound of formula (I) is for example a base-addition salt. A base-addition salt is formed by bringing the compound of formula (I) into contact with a suitable organic or inorganic base. A base addition salt may be formed using a suitable organic base like a nitrogen base, for example ammonia or a trialkylamine like triethylamine. A base addition salt may also for example be formed using a suitable inorganic base like an alkali metal or rare earth hydroxide, for example potassium hydroxide, sodium hydroxide, magnesium hydroxide or manganese hydroxide.

In one embodiment there is provided a compound of formula (I) which is a free acid.

In one embodiment there is provided a compound of formula (I) which is a salt.

In one embodiment there is provided a compound of formula (I) which is a sodium, potassium, magnesium, or ammonium salt.

In one embodiment there is provided a compound of formula (I) which is a sodium, potassium, or ammonium salt.

In one embodiment there is provided a compound of formula (I) which is a sodium or ammonium salt.

In one embodiment there is provided a compound of formula (I) which is a sodium salt.

In one embodiment there is provided a compound of formula (I) which is a monopotassium, dipotassium, tripotassium, tetrapotassium, monosodium, disodium, trisodium, tetrasodium, monoammonium, diammonium, triammonium or tetraammonium salt.

In one embodiment there is provided a compound of formula (I) which is a monosodium, disodium, trisodium, tetrasodium, monoammonium, diammonium, triammonium or tetraammonium salt.

In one embodiment there is provided a compound of formula (I) which is a monosodium, disodium, trisodium or tetrasodium salt.

In one embodiment there is provided a compound of formula (I) which is a monosodium salt.

In one embodiment there is provided a compound of formula (I) which is a disodium salt.

In one embodiment there is provided a compound of formula (I) which is a trisodium salt.

In one embodiment there is provided a compound of formula (I) which is a monoammonium, diammonium, triammonium or tetraammonium salt.

In one embodiment there is provided a compound of formula (I) which is a monoammonium salt.

In one embodiment there is provided a compound of formula (I) which is a diammonum salt.

In one embodiment there is provided a compound of formula (I) which is a triammonium salt.

In one embodiment there is provided any compound of formula (I), physiologically cleavable precursor or salt thereof disclosed in the Examples.

### Synthetic Processes

In one embodiment there is provided a process for preparing a compound of formula (I) a physiologically cleavable precursor or a salt thereof comprising:
i. Providing a compound of formula (V) or a salt thereof:

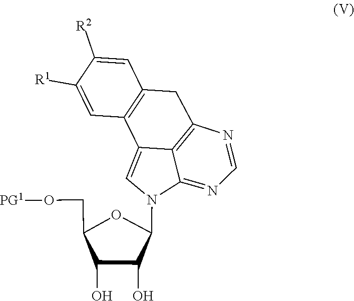

Where R¹ is selected from hydro and R² is selected from cyano, or R¹ and R² together with the atoms to which they are attached form a 6-membered carboaromatic ring, and PG¹ is a suitable protecting group;
ii. Immobilising the compound of formula (II) or a salt thereof by linking one of its secondary alcohol groups to a suitable support;
iii. Capping any remaining secondary alcohol groups with a suitable protecting group PG²;
iv. Removing the protecting group PG¹;
V. Reacting the exposed primary alcohol group with a compound of formula (VI):

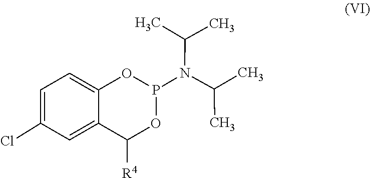

Where R⁴ is selected from a hydro group and a C₁₋₃alkyl group;
vi. Oxidising the resultant phosphorus (III) compound to a phosphorus (V) compound; optionally
vii. Reacting the phosphorus (V) compound with a tetraalkylammonium pyrophosphate to generate a triphosphate;
viii. Removing the protecting group PG²;
ix. Cleaving the resultant phosphate from the support to generate a compound of formula (I) or salt thereof; and optionally
x. Forming a free acid, physiologically cleavable precursor or different salt of the compound of formula (I).

In one embodiment there is provided a process for preparing a compound of formula (I), a physiologically cleavable precursor or a salt thereof comprising:
i. Providing a compound of formula (V) or a salt thereof:

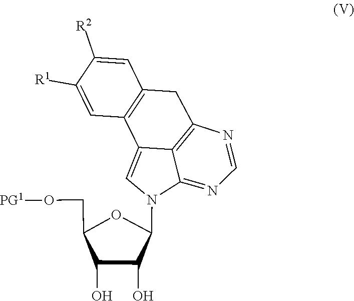

Where R¹ is selected from hydro and R² is selected from cyano, or R¹ and R² together with the atoms to which they are attached form a 6-membered carboaromatic ring, and PG¹ is a suitable protecting group;
ii. Immobilising the compound of formula (II) or a salt thereof by linking one of its secondary alcohol groups to a suitable support;
iii. Capping any remaining secondary alcohol groups with a suitable protecting group PG²;
iv. Removing the protecting group PG¹;
v. Reacting the exposed primary alcohol group with a compound of formula (VI):

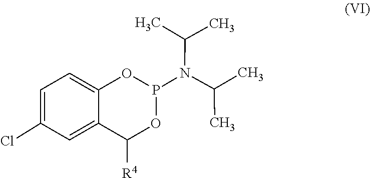

Where R⁴ is selected from a hydro group and a C₁₋₃alkyl group;
vi. Oxidising the resultant phosphorus (III) compound to a phosphorus (V) compound;
vii. Reacting the phosphorus (V) compound with a tetraalkylammonium pyrophosphate to generate a triphosphate;
viii. Removing the protecting group PG²;
ix. Cleaving the resultant triphosphate from the support to generate a compound of formula (I) or salt thereof; and optionally
xi. Forming a free acid, physiologically cleavable precursor or different salt of the compound of formula (I).

A protecting group (“PG”, for example PG¹ or PG²) is any group suitable for temporarily protecting a reactive centre, for example a hydroxyl group. Suitable protecting groups for the reactive centres disclosed herein may be found for example in “Greene's Protective Groups in Organic Synthesis, Fourth Edition”, Greene T. W., Wuts P. G. M.; John Wiley & Sons, Inc. 2007, doi:10.1002/0470053488), the contents of all of which are herein incorporated by reference.

A “C₁₋₃alkyl group” is a straight chain or branched saturated alkyl group with the indicated number of carbons. Example C₁₋₃alkyl groups include methyl, ethyl, propyl and isopropyl.

In step iii) above, the secondary alcohols to be capped may be those on the ribose part of the molecule.

This overall process is an advantageous preparation of the compound of formula (I) for several reasons:
There is no need for prior protection of the secondary alcohol groups on the starting protected nucleoside;
After step 1, unreacted nucleoside can be recovered, which minimizes loss of material;
The solid-supported nucleosides can be stored for up to 3 months without degradation;
The route is compatible with automated synthesis;
The process is robust and has good overall reproducibility;
Clean phosphorylation crude products are generated that are easy to purify; and
Gives high phosphorylation yields (typically ca. 60% starting from the nucleoside loaded resin).

In some embodiments R⁴ may be a hydro group.

In some embodiments R⁴ may be a C₁₋₃alkyl group. It has been observed that when R¹ is a C₁₋₃alkyl group, the phosphoramidite reagent preparation is easier and higher yielding, but performs at least as well in step v above as when R⁴ is a hydro group.

In some embodiments R⁴ may be methyl.

In one embodiment there is provided a compound of formula (VI):

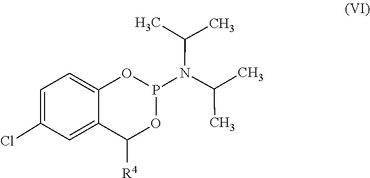

Where R⁴ is a C₁₋₃ alkyl group.

In one embodiment there is provided a compound of formula (Vla):

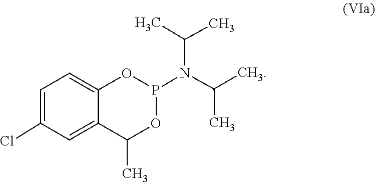 [SMILES: CC1OP(N(C(C)C)C(C)C)Oc2ccc(Cl)cc21]

In some embodiments the support may be a solid polymer.

In some embodiments the support may be a solid polymer selected from controlled-porosity glass and polystyrene.

In some embodiments the support may be polystyrene.

In some embodiments the support may be controlled-porosity glass.

In some embodiments the support may be functionalised with a primary amino group. This may form the reactive point of attachment to the support.

In some embodiments the support may be controlled-porosity glass functionalised with a

primary amino group (for example Amino-SynBase™).

In some embodiments PG¹ may be selected from trityl, dimethoxytrityl and trimethoxytrityl.

In some embodiments PG² may be selected from acetyl, benzoyl, 2,2,2-trichloroethylcarbonyl, paramethoxybenzyl, methyl, tetrahydropyranyl, triethylsilyl, triisopropylsilyl, trimethylsilyl, tert-butyldimethylsilyl and methoxyethyl.

In some embodiments PG² may be acetyl. Where an immobilised molecule is base labile, this allows for an efficient synthesis in which removal of the PG² group and cleavage from the resin may be accomplished in a single step.

In some embodiments PG¹ may be dimethoxytrityl and PG² may be acetyl.

In some embodiments immobilisation of the compound of formula (II) in step i) may occur mainly at the 2′-hydroxy position.

When immobilisation occurs mainly at the 2′-hydroxy position, this may be >50%, >60%, >70%, >80%, >90% or 100% of the total immobilisation (i.e. the total covalent binding of both secondary hydroxyl groups to the support).

In some embodiments the tetraalkylammonium pyrophosphate may be tetrabutylammonium pyrophosphate.

In one embodiment there is provided a process for preparing a compound of formula (I), a physiologically cleavable precursor or a salt thereof comprising:
i. Providing a compound of formula (II) or a salt thereof:

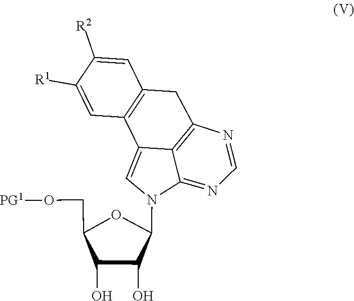

Where R¹ is selected from hydro and R² is selected from cyano, or R¹ and R² together with the atoms to which they are attached form a 6-membered carboaromatic ring, and PG¹ is selected from trityl, dimethoxytrityl and trimethoxytrityl;
ii. Immobilising the compound of formula (V) or a salt thereof by linking one of its secondary alcohol groups to a controlled-porosity glass support;
iii. Capping any remaining secondary alcohol groups with a protecting group PG² selected from acetyl, benzoyl, 2,2,2-trichloroethylcarbonyl, paramethoxybenzyl, methyl, tetrahydropyranyl, triethylsilyl, triisopropylsilyl, trimethylsilyl, tert-butyldimethylsilyl and methoxyethyl;
iv. Removing the protecting group PG¹;
v. Reacting the exposed primary alcohol group with a compound of formula (VI):

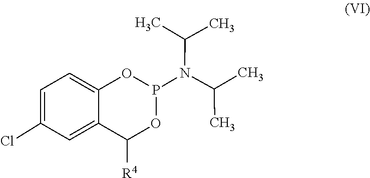

Where R⁴ is a C₁₋₃alkyl group;
vi. Oxidising the resultant phosphorus (III) compound to a phosphorus (V) compound; optionally
vii. Reacting the phosphorus (V) compound with tetrabutylammonium pyrophosphate to generate a triphosphate;
viii. Removing the protecting group PG²;
ix. Cleaving the resultant phosphate from the support to generate a compound of formula (I) or salt thereof; and optionally
x. Forming a free acid, physiologically cleavable precursor or different salt of the compound of formula (I).

In one embodiment there is provided a process for preparing a compound of formula (I), a physiologically cleavable precursor or a salt thereof comprising:
i. Providing a compound of formula (V) or a salt thereof:

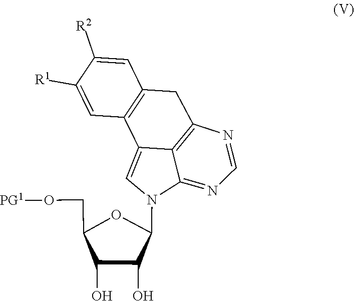

Where R¹ is selected from hydro and R² is selected from cyano, or R¹ and R² together with the atoms to which they are attached form a 6-membered carboaromatic ring, and PG¹ is dimethoxytrityl;
ii. Immobilising the compound of formula (II) or a salt thereof by linking one of its secondary alcohol groups to a controlled-porosity glass support;
iii. Capping any remaining secondary alcohol groups with a protecting group PG² which is acetyl;
iv. Removing the protecting group PG¹;
v. Reacting the exposed primary alcohol group with a compound of formula (VI):

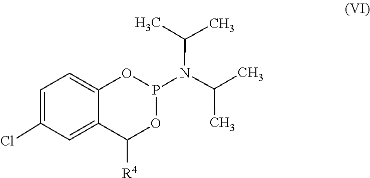

Where R⁴ is a methyl group;
vi. Oxidising the resultant phosphorus (III) compound to a phosphorus (V) compound; optionally
vii. Reacting the phosphorus (V) compound with tetrabutylammonium pyrophosphate to generate a triphosphate;
viii. Removing the protecting group PG²;
ix. Cleaving the resultant phosphate from the support to generate a compound of formula (I) or salt thereof; and optionally
x. Forming a free acid, physiologically cleavable precursor or different salt of the compound of formula (I).

Suitable conditions and reagents to effect each of steps i) to ix) above are known to the skilled person or can be found in the Detailed Description.

In some embodiments immobilising the compound of formula (II) or salt thereof in step ii) may be accomplished by a coupling reagent (for example succinic anhydride catalysed by dimethylaminopyridine when the support is functionalised with a primary amino group).

In some embodiments reaction of the exposed primary alcohol group with a compound of formula (III) may be accomplished using an activator (for example BTT activator or Activator 42®).

In some embodiments the phosphorus (III) compound in step vi) may be oxidised to a phosphorus(V) compound using aqueous pyridine and iodine.

In some embodiments cleaving the triphosphate from the support may be accomplished using basic conditions (for example by treating with AMA). When there is a base-labile support and a base-labile protecting group is chosen for PG², using these conditions allows simultaneous deprotection and cleavage.

### RNA Synthesis Compositions

Compounds of formula (I) may be used as substrates for RNA synthesis along with other natural and synthetic RNA building blocks.

In one embodiment there is provided a composition for preparing a labelled RNA molecule comprising a compound of formula (I) and a natural ribonucleotide.

A “natural ribonucleotide” comprises the appropriate natural ribonucleoside with a phosphate group (for example a monophosphate, diphosphate, or triphosphate group, such as those described by the definition of R³ herein) bonded to the 5′ hydroxy position. In some embodiments a “natural ribonucleotide” means a natural ribonucleoside triphosphate.

In some embodiments a natural ribonucleotide (for example a natural ribonucleoside triphosphate) may be selected from cytidine 5′-triphosphate, uridine 5′-triphosphate, adenosine 5′-triphosphate and guanidine 5′-triphosphate. A composition of natural ribonucleotides (for example a composition of natural nucleoside triphosphates) may comprise combinations of varying amounts of these building blocks, in amounts sufficient to construct a target RNA molecule (for example as provided in NTP mix).

### Labelled RNA Synthesis

In one embodiment there is provided the use of a compound of formula (I), a physiologically cleavable precursor or a salt thereof to prepare a labelled RNA molecule (for example a 2CNqA or pA labelled RNA molecule).

A labelled RNA molecule comprises at least one modified fluorescent residue (for example a residue derived from a compound of formula (I) such that the modified residue is a 2CNqA or pA residue) but is otherwise similar to the natural RNA molecule (i.e. one with an unmodified adenosine residue at the same location as the 2CnqA or pA residue).

In some embodiments the labelled RNA molecule may be a 2CNqA or pA labelled mRNA (messenger RNA) molecule.

In one embodiment there is provided the use of a compound of formula (I), a physiologically cleavable precursor or a salt thereof where R¹ is selected from hydro and R² is selected from cyano to prepare an RNA molecule labelled with 2CNqA.

In one embodiment there is provided the use of a compound of formula (I), a physiologically cleavable precursor or a salt thereof where R¹ and R² together with the atoms to which they are attached form a 6-membered carboaromatic ring to prepare an RNA molecule labelled with pA.

In one embodiment there is provided the use of a compound of formula (I), a physiologically cleavable precursor or a salt thereof to enzymatically prepare a labelled RNA molecule.

In one embodiment there is provided the use of a compound of formula (I), a physiologically cleavable precursor or a salt thereof to enzymatically prepare a labelled RNA molecule in-vitro.

In one embodiment there is provided a process for preparing a labelled RNA molecule in-vitro comprising:
i. Providing a DNA template to a composition comprising a compound of formula (I), a physiologically cleavable precursor or a salt thereof and a natural ribonucleotide;
ii. Treating the resultant mixture with an RNA polymerase; optionally
iii. Monitoring the labelled RNA molecule using microscopy; and optionally
iv. Isolating the labelled RNA molecule.

In one embodiment there is provided a process for preparing a labelled RNA molecule in-vitro comprising:
v. Providing a DNA template to a composition comprising a compound of formula (I), a physiologically cleavable precursor or a salt thereof and a natural ribonucleotide;
vi. Treating the resultant mixture with an RNA polymerase;
vii. Monitoring the labelled RNA molecule using microscopy; and
viii. Isolating the labelled RNA molecule.

In some embodiments microscopy may be confocal laser scanning fluorescence microscopy.

In some embodiments a process for preparing a labelled RNA molecule in-vitro may be carried out in the presence of transcription buffer (e.g. 5X transcription buffer), magnesium salt (e.g. magnesium(II) chloride) and/or an RNase inhibitor (e.g. Ribolock).

In one embodiment there is provided a kit for preparing a labelled RNA molecule comprising:
i. A compound of formula (I);
ii. A composition of natural ribonucleotides;
iii. An RNA polymerase; optionally
iv. A DNA template; and optionally
v. Instructions for use.

In one embodiment there is provided the use of a compound of formula (I), a physiologically cleavable precursor or a salt thereof to prepare an endogenously labelled RNA molecule.

In one embodiment there is provided the use of a compound of formula (I), a physiologically cleavable precursor or a salt thereof in live cells to prepare an endogenously labelled RNA molecule.

In one embodiment there is provided the use of a compound of formula (I), a physiologically cleavable precursor or a salt thereof to prepare a labelled RNA molecule in-cellulo.

In one embodiment there is provided a process for preparing a labelled RNA molecule in-cellulo comprising:
i. Providing a compound of formula (I), a physiologically cleavable precursor or a salt thereof to a eukaryotic or prokaryotic cell;
ii. Allowing the organism to spontaneously internalise the compound;
iii. Allowing the organism to prepare a labelled RNA molecule; optionally
iv. Monitoring the labelled RNA molecule using microscopy; and optionally
v. Isolating the labelled RNA molecule.

In one embodiment there is provided a process for preparing a labelled RNA molecule in-cellulo comprising:
vi. Providing a compound of formula (I), a physiologically cleavable precursor or a salt thereof to a eukaryotic or prokaryotic cell;
vii. Allowing the organism to spontaneously internalise the compound;
viii. Allowing the organism to prepare a labelled RNA molecule;
ix. Monitoring the labelled RNA molecule using microscopy; and
x. Isolating the labelled RNA molecule.

In some embodiments a eukaryotic cell may be comprised in c. elegans or a zebra fish.

In some embodiments a labelled RNA molecule may comprise >10%, >20%, >30%, >40%, >50%, >60%, >70%, >80%, >90% or 100% of modified residues (for example derived from a compound of formula (I) such that the modified residue is a 2CNqA or pA residue) in place of unmodified adenosine residues.

In some embodiments a labelled RNA molecule may comprise 10%-20%, 10%-30%, 10%-40%, 20%-50%, 30%-60%, 40%-70%, 50%-80% or 50%-90% of modified residues (for example derived from a compound of formula (I) such that the modified residue is a 2CNqA or pA residue) in place of unmodified adenosine residues.

In some embodiments a labelled RNA molecule may be selected from mRNA and ribosomal RNA.

In one embodiment there is provided a process for preparing a labelled RNA molecule in-cellulo essentially as described in the detailed description.

### FIGURES

FIG. 1: General scheme for preparation of compounds of formula (I).

FIG. 2: Preparation of pA nucleoside.

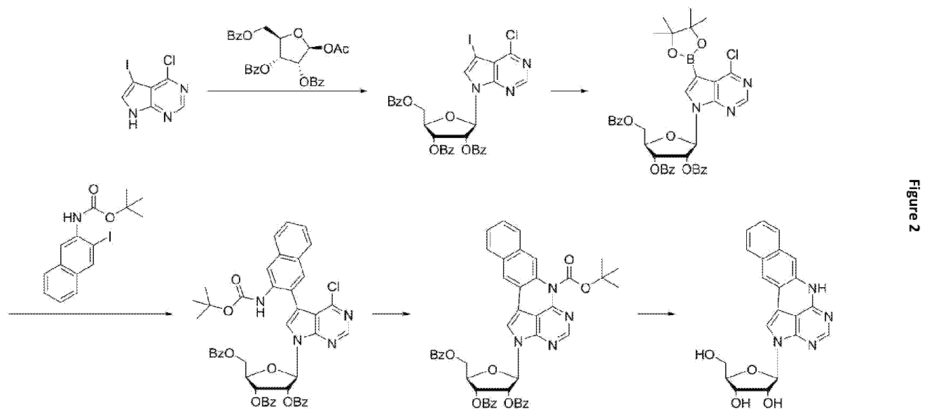

FIG. 3: Cytotoxicity assessment. (A) Cell viability measured as reduction in metabolic activity using the alamarBlue assay (B) Cell membrane integrity assessment measured by the lactate dehydrogenase (LDH) leakage assay. Huh-7 cells were treated with the compounds for 24 hours at the indicated dose. Error bars represent standard deviation of three independent experiments.

FIG. 4: Confocal fluorescence microscopy images of live Huh-7 cells exposed to 2.5 μM 2CNqATP or pATP in complete cell culture medium for (A) 20 h at 37° C. (B) 1.5 h at 37° C. or (C) 1.5 h at 4° C.

FIG. 5: Measured mean fluorescence intensity (MFI) of 2CNqATP or pATP inside single living cells after exposure to 2.5 μM pATP or 2CNqATP after the indicated time. Cells were washed, trypsinized and analysed using flow cytometry with excitation at 405 nm. A) MFI distribution of the measured cell samples for non-treated cells, and cells treated with 2CNqATP and pATP. B) Mean MFI of 2CNqATP in single living cells plotted against the exposure time. C) Mean MFI of pATP in single living cells plotted against the exposure time. Lines are to guide the eyes.

FIG. 6: Dose response of cell uptake measured as normalized fluorescence intensity in cell lysates harvested from Huh-7 cell cultures exposed to different concentrations of A) 2CNqATP or B) pATP for 24 h. 2CNqATP was excited at 355 nm and emission detected using 460 nm bandpass filter. For pATP bandpass filter for excitation at 380 nm and emission at 410 nm were used. Error bars represent standard deviation.

FIG. 7: Normalized mean fluorescence intensity of live Huh-7 cells after exposure to 2.5 μM 2CNqATP (upper graph) or pATP (lower graph) in presence of increasing concentrations of ATP (black, solid connecting line) or adenosine (grey, dotted connecting line). Cells were exposed for 4 h, washed and analysed by flow cytometry using 405 nm laser for excitation. Lines are to guide the eyes; error bars represent standard deviation of three independent experiments. Lines are to guide the eyes.

FIG. 8: Fluorescence emission spectra of cell-extracted and purified RNA from Huh-7 cells treated with 2.5 μM of (A) 2CNqATP or (B) pATP for 24 h showing that cell machinery is active and can incorporate certain nucleotide analogues into endogenously produced RNA. Black solid lines represent cell-extracted labelled RNA; grey dotted lines represent the following controls: (light black, dashed) compound added to cell-lysate of non-treated cells prior to RNA purification; compound added to RNA prior to final column purification (dark grey, dotted); and compound added directly to the RNA purification column (light grey, dashed). The spectra are normalized to the corresponding absorption at 260 nm, reflecting the total RNA concentration in the solutions.

FIG. 9: Spectral comparison of extracted RNA from 2CNqATP-treated Huh-7 cells to 2CNqATP and in-vitro 2CNqA-modified RNA strands. A) Normalized excitation spectrum of extracted RNA from Huh-7 cells that were exposed to 2.5 μM 2CNqATP over 24 h (black, thick line) overlaid with absorption spectra of 2CNqATP (grey, dashed line) and 2CNqA incorporated in 25:mers (5′-CGA CAA AAU CAA [2CNqA]AU GCG UGA UUG G-3′) of ssRNA (black, thin line) or, with the hybridized complementary strand, dsRNA (grey, thin line). Each spectrum is normalized to the peak maximum. To record excitation spectra, the emission wavelength was fixed at 443 nm. B) Normalized emission spectra of extracted RNA from cells that were exposed to 2.5 μM 2CNqATP (black, thick line), of 2CNqATP (grey, dashed line), 2CNqA incorporated in 25:mer ssRNA (as in A) (black, thin line) and dsRNA (grey, thin line).

FIG. 10: Cytotoxicity assessment using 2CNqAMP. (A) Cell viability measured as reduction in metabolic activity using the alamarBlue assay (B) Cell membrane integrity assessment measured by the lactate dehydrogenase (LDH) leakage assay. Huh-7 cells were treated with the compounds for 24 hours at the indicated dose. Error bars represent standard deviation of three exposures.

FIG. 11: Confocal fluorescence microscopy images of live Huh-7 cells exposed to 2.5 μM 2CNqAMP or DPBS (as control) in complete cell culture medium for (A) 24 h at 37° C. (B) 1.5 h at 37° C. or (C) 1.5 h at 4° C.

FIG. 12: Measured mean fluorescence intensity (MFI) of 2CNqAMP inside single living cells after exposure to 2.5 μM 2CNqAMP after the indicated time. Cells were washed, trypsinized and analysed using flow cytometry with excitation at 405 nm. Mean MFI of 2CNqAMP in single living cells plotted against the exposure time. Shown are two independent experiments (indicated as empty/filled squares). Error bars represent standard deviation of three exposures.

FIG. 13: Spectroscopic readout of cell-extracted and purified RNA from Huh-7 cells treated with 2.5 μM 2CNqAMP for 24 h at 37° C. showing that cell machinery is active and can incorporate certain nucleotide analogues into endogenously produced RNA. (A) Spectral comparison of extracted RNA from 2CNqAMP-treated Huh-7 cells to 2CNqATP, RNA from 2CNqATP-treated Huh-7 cells, and in-vitro 2CNqA-modified RNA strands. Excitation spectrum of extracted RNA from Huh-7 cells that were exposed to 2.5 μM 2CNqAMP over 24 h (black, solid line) overlaid with absorption spectra of 2CNqATP (black, dashed line), 2CNqA incorporated in 25:mers (5′-CGA CAA AAU CAA [2CNqA]AU GCG UGA UUG G-3′) of ssRNA (grey, dashed-dot line) or, with the hybridized complementary strand, dsRNA (grey, dashed line), and excitation spectrum of RNA from 2CNqATP-treated Huh-7 cells (grey, solid line). Each spectrum is normalized to the peak maximum. To record excitation spectra, the emission wavelength was fixed to 443 nm. (B) Fluorescence emission spectra of cell-extracted and purified RNA from Huh-7 cells treated with 2.5 μM of 2CNqAMP for 24 h. Black solid lines represent cell-extracted labelled RNA; grey solid line represents a control where compound is added to cell-lysate of non-treated cells prior to RNA purification. The spectra are normalized to the corresponding absorption at 260 nm, reflecting the total RNA concentration in the solutions. Light grey dashed line presents the emission spectrum of ultrapure water (milliQ) for comparison.

### DETAILED DESCRIPTION

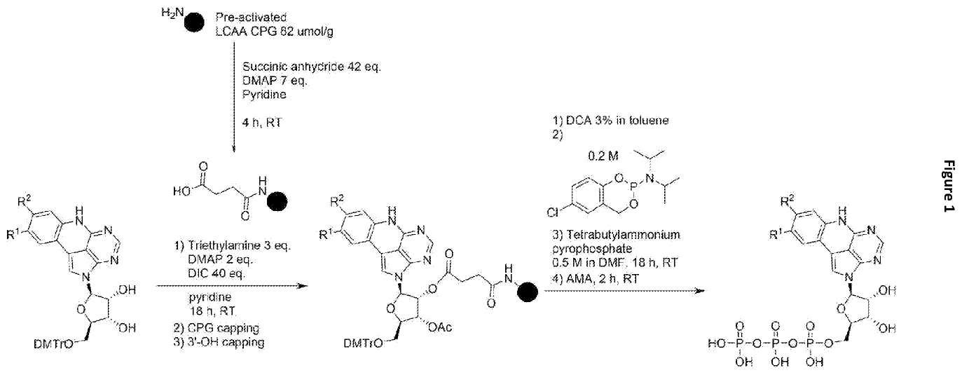

Compounds of formula (I) may be prepared according to the synthetic scheme shown in FIG. 1 and the methods described below. Unless otherwise noted, reagents were commercially available and used without further purification. The following reagents used for the triphosphorylation were bought from Sigma-Aldrich: DCA deblock for ÄKTA, CAP A for ÄKTA, CAP B1 and B2 for ÄKTA, BTT Activator. ¹H (500 MHz) and ¹³C (126 MHz) NMR spectra were recorded at 300 K on a Bruker 500 MHz system equipped with a CryoProbe. ³¹P (202 MHz) NMR spectra were recorded at 300 K on a Bruker 500 MHz system. All shifts are recorded in ppm relative to the deuterated solvent (DMSO-d6, CDCl₃ or D₂O).

### Example 1: Synthesis of Modified Nucleoside Phosphates—2CNqATP

2-(5-O-(4,4′-Dimethoxytrityl)-β-D-ribofuranosyl)-2,6-dihydro-2,3,5,6-tetraazaaceanthrylene-8-carbonitrile 1

Compound 1 was prepared according to the literature (Wypijewska del Nogal et al., Nucleic Acids Research 48, 7640-7652 [2020]). Analytical data were consistent with those reported.

### CPG Solid Support 3

Amino-SynBase™ CPG 500/110 (LCAA) 2 from LinkTech (Nu. 1397-C025, 1 g, 0.08 mmol) was activated by shaking in trichloroacetic acid 3% in DCE (8 mL, 0.08 mmol) for 18 h. The activated support was then filtered off and washed with 9:1 triethylamine:diisopropylethylamine (20 mL), dichloromethane (20 mL) and diethyl ether (20 mL). The activated support was dried under vacuum for 2 days before use. Subsequently, the support (1 g, 0.08 mmol), succinic anhydride (0.345 g, 3.44 mmol) and N,N-dimethylpyridin-4-amine (0.070 g, 0.57 mmol) were suspended in dry Pyridine (3 mL) under N₂. The reaction mixture was then gently shaken at RT for 4 h. After 4 h, solvent was filtered off and the support washed successively with pyridine (20 mL), dichloromethane (20 mL), diethyl ether (20 mL) and air-dried. Negative ninhydrin test on a small portion of support proved full succinylation. Succinylated CPG 3 could thereafter be kept at room temperature for several months.

CPG-supported 2-(5-O-(4,4′-Dimethoxytrityl)-β-D-ribofuranosyl)-2,6-dihydro-2,3,5,6-tetraazaaceanthrylene-8-carbonitrile 4

In a 10 mL syringe with PTFE filter, succinylated support 3 (0.400 g, 82 μmol/g, 0.03 mmol), DMAP (8 mg, 0.07 mmol), DIC (203 μl, 1.31 mmol), nucleoside 1 (0.022 g, 0.03 mmol) and triethylamine (14 μl, 0.10 mmol) were suspended pyridine (3 mL). The mixture was gently shaken for 18 h at RT. After 18 h, the syringe was purged and the support washed with pyridine (5 mL), dichloromethane (5 mL) and diethyl ether. Subsequently, in the same syringe, DMAP (8 mg, 0.07 mmol), DIC (203 μl, 1.31 mmol), triethylamine (14 μl, 0.10 mmol) and 2,3,4,5,6-pentachlorophenol (0.87 g, 0.33 mmol) were added to the support and suspended in pyridine (3 mL). The mixture was gently shaken for 4 h at RT before a solution of piperidine (2 mL, 20% in DMF—for capping of the unreacted carboxylic acids on the support) was added for 1 min (longer exposure time will reduce loading as piperidine cleaves the ester bonds with the nucleoside), then quickly washed away with DMF (3×5 mL), dichloromethane (5 mL) and diethyl ether (5 mL). Finally, the resin was shaken in a CAP A+CAP B mix (50/50 v/v) for 2 hours under argon atmosphere, then washed with DMF (5 mL), dichloromethane (5 mL), diethyl ether (5 mL) and argon-dried (final loading: 16 μmol/g−determined by reading optical density of a DMT solution cleaved from a weighed amount of support−ε=70000 M−1.cm−1 at 498 nm). Final loading can be increased by performing a second coupling with 1 in the same conditions before capping (typical loading after second coupling 20-25 82 mol/g). Concentrating the reaction mixture and washing the residue multiple times with water and diethyl ether allows recovery of nearly 85% of unreacted nucleoside 1.

6-chloro-N,N-diisopropyl-4H-benzo[d][1,3,2]dioxaphosphinin-2-amine 5

Compound 5 was prepared according to the literature (Ducho, C. et al., J. Med. Chem. 50, 1335-1346 [2007]). Briefly, 5-chlorosalicylic acid was reduced with LAH (0.5 equiv.) at −20° C. and the resulting 5-chlorosalicylic alcohol was cyclized into 2,6-dichloro-4H-benzo[d][1,3,2]dioxaphosphinine using PCl₃ (1.2 equiv.) and triethylamine (2.3 equiv.) at −20° C. under argon. Low temperature and use of triethylamine as the base were decisive in avoiding rapid and quantitative Arbuzov rearrangement of the desired product into the more stable 2,5-dichloro-3H-benzo[d][1,2]oxaphosphole 2-oxide. The crude 2,6-dichloro-4H-benzo[d][1,3,2]dioxaphosphinine was subsequently treated with diisopropylamine (3 equiv.) for 2 h at room temperature. The mixture was then filtered under argon, concentrated to dryness and taken in 20% diisopropylamine in heptane. Quick filtration on a small silica gel plug allowed desired compound 5 as a colourless oil, crystallizing over time at −20° C. Any attempt of more thorough column chromatography on compound 5 would lead to quantitative Arbuzov rearrangement.

¹H NMR (500 MHz, DMSO-d6) δ=7.23 (dd, J=8.6, 2.6 Hz, 1H), 7.20 (d, J=2.4 Hz, 1H), 6.92 (d, J=8.6 Hz, 1H), 5.06 (dd, J=14.7, 5.2 Hz, 1H), 4.89 (dd, J=19.6, 14.8 Hz, 1H), 3.53-3.63 (m, 2H), 1.15-1.19 (dd, J=8.0, 7.0 Hz, 12H). 31P NMR (202 MHz, DMSO-d6) δ=136.00 (s, 1P).

Bis(tetrabutylammonium) dihydrogen diphosphate 6

Compound 6 was prepared according to the literature (Warnecke, S. & Meier, C., J. Org. Chem. 74, 3024-3030 [2009]).

¹H NMR (500 MHz, D₂O) δ 3.04-3.13 (m, 16H), 1.53 (bs, 16H), 1.24 (h, J=7.3, 16H), 0.83 (t, J=7.4, 24H). ³¹P NMR (202 MHz, D₂O) δ=−10.78 (s, 2P).

((2R,3S,4R,5R)-5-(8-cyano-2,3,5,6-tetraazaaceanthrylen-2(6H)-yl)-3,4-dihydroxytetrahydrofuran-2-yl)methyl hydrogen triphosphate (2CNqATP) 7

Reaction was performed in a 5 ml syringe with PTFE filter loaded with the CPG-bound 2CNaA nucleoside 4 (400 mg, 0.0064 mmol) under an argon atmosphere and shaking. Steps were performed as following:
a. 5′-DMT removal: the support was washed with a flow of DCA deblock until the filtrate was colorless, then washed with ACN (5×5 mL).
b. Coupling: N,N-diisopropyl-4H-benzo[d][1,3,2]dioxaphosphinin-2-amine 5 (345 mg, 1.36 mmol) was dissolved in 4.8 mL ACN and reacted portionwise with the support (3 equal couplings with reaction times 60 s-60 s-90 s respectively). To each coupling, BTT activator (2.4 mL) was also added. The support was subsequently washed with ACN (3×5 mL).
c. Oxidation: Pyridine/Water/lodine (9/1/12.7 v/v/w, 5 mL) for 45 s, followed by ACN wash (3×5 mL) and drying of the support in an argon flow.
d. Triphosphorylation: Two injections of bis(tetrabutylammonium) dihydrogen diphosphate 6 (0.5 M, 5 ml) for 15 min and 18 hours, respectively. The support was subsequently rinsed with DMF (5 mL), water (3×5 mL), ACN (5 mL) and then dried in an argon flow.
e. Cleavage and Purification: Cleavage of the triphosphate was done in 2 h at room temperature with AMA (50/50 v/v mix of 23% aq. NH4OH and 40% aq. methylamine, 5 mL). After 2 hours, the AMA filtrate was purged in a round-bottom flask and the support was rinsed 3 times with 23% aq. NH₄OH solution. After freeze-drying of the mixture, purification by HPLC (Waters Acquity HSS T3 column, 2.1×50 mm, 0.4 mL/min, 2 to 99% 50 mM NH₄OAc in water 80:20 EtOH) was performed to furnish 2CNqATP (2.3 mg, 55% determined from UV absorbance) as a light-yellow solid (ammonium salt).

HRMS (ESI-TOF) m/z calcd. for C₁₈H₁₉N₅O₁₃P₃ [M+H]+: 606.0192, found: 606.0170.

((2R,3S,4R,5R)-5-(8-cyano-2,3,5,6-tetraazaaceanthrylen-2(6H)-yl)-3,4-dihydroxytetrahydrofuran-2-yl)methyl hydrogen triphosphate can also be made by a slightly modified route wherein the coupling step (b above) is carried out with a modified phosphoramidite such as 6-chloro-N, N-diisopropyl-4-methyl-4H-benzo[d][1,3,2]dioxaphosphinin-2-amine 8 (compound (IIIa) above). This reagent has been found to be more easily prepared: compound 8 is obtainable in a yield of 60% compared to around 3-10% for the preparation of compound 5 under the conditions in this specification.

### Example 1a: Synthesis of Modified Nucleoside Phosphates—2CNqAMP

((2R,3S,4R,5R)-5-(8-cyano-2,3,5,6-tetraazaaceanthrylen-2(6H)-yl)-3,4-dihydroxytetrahydrofuran-2-yl)methyl dihydrogen phosphate 7a

Reaction was performed in a 5 ml syringe with PTFE filter loaded with the CPG-bound 2CNaA nucleoside 4 prepared above (400 mg, 0.0064 mmol) under an argon atmosphere and shaking. Steps were performed as following:
a. 5′-DMT removal: the support was washed with a flow of DCA deblock until the filtrate was colorless, then washed with ACN (5×5 mL).
b. Coupling: N,N-diisopropyl-4H-benzo[d][1,3,2]dioxaphosphinin-2-amine 5 (345 mg, 1.36 mmol) was dissolved in 4.8 mL ACN and reacted portionwise with the support (3 equal couplings with reaction times 60 s-60 s-90 s respectively). To each coupling, BTT activator (2.4 mL) was also added. The support was subsequently washed with ACN (3×5 mL).
c. Oxidation: Pyridine/Water/lodine (9/1/12.7 v/v/w, 5 mL) for 45 s, followed by ACN wash (3×5 mL) and drying of the support in an argon flow.
d. Cleavage and Purification: Cleavage of the triphosphate was done in 2 h at room temperature with AMA (50/50 v/v mix of 23% aq. NH₄OH and 40% aq. methylamine, 5 mL). After 2 hours, the AMA filtrate was purged in a round-bottom flask and the support was rinsed 3 times with 23% aq. NH4OH solution. After freeze-drying of the mixture, purification by HPLC (Waters Acquity HSS T3 column, 2.1×50 mm, 0.4 mL/min, 2 to 99% 50 mM NH₄OAc in water 80:20 EtOH) was performed to furnish 2CNqAMP (2.3 mg, 55% determined from UV absorbance) as a light-yellow solid (ammonium salt).
   6-chloro-N,N-diisopropyl-4-methyl-4H-benzo[d][1,3,2]dioxaphosphinin-2-amine 8

5-chloro-2-hydroxybenzaldehyde was reacted with methylmagnesium bromide (2.5 equiv.) at −20° C. and the resulting 4-chloro-2-(1-hydroxyethyl)phenol was cyclized into 2,6-dichloro-4-methyl-4H-benzo[d][1,3,2]dioxaphosphinine using PCl3 (1.2 equiv.) and triethylamine (2.3 equiv.) at −20° C. under argon. The crude 2,6-dichloro-4-methyl-4H-benzo[d][1,3,2]dioxaphosphinine was subsequently treated with diisopropylamine (3 equiv.) for 2 h at room temperature. The mixture was then filtered under argon, concentrated to dryness and taken in 20% diisopropylamine in heptane. Quick filtration on a small silica gel plug furnished desired compound 8 as a colourless oil.

¹H NMR (500 MHz, DMSO-d6) δ=6.96 (d, J=8.5 Hz, 1H), 6.87 (d, J=8.5 Hz, 1H), 6.74 (d, J=8.4 Hz, 1H), 5.19-5.26 (m, 1H), 5.16 (dq, J=10.4, 6.6 Hz, 1H), 3.57 (tdt, J=13.6, 10.6, 6.8 Hz, 2H), 1.63 (d, J=6.6 Hz, 3H), 1.55 (d, J=6.4 Hz, 2H), 1.16-1.19 (m, 24H). ³¹P NMR (202 MHz, DMSO-d6) δ=137.63 (s, 1P), 127.90 (s, 1P).

### Example 2: Synthesis of Modified Nucleoside Phosphates—pATP

(2R,3R,4R,5R)-2-((Benzoyloxy)methyl)-5-(4-chloro-5-iodo-7H-pyrrolo[2,3-d]pyrimidin-7-yl)tetrahydrofuran-3,4-diyl dibenzoate 9

4-chloro-5-iodo-7H-pyrrolo[2,3-d]pyrimidine (20 g, 71.6 mmol) was dissolved in MeCN (480 mL). trimethylsilyl (E)-N-(trimethylsilyl)acetimidate (19.5 mL, 78.7 mmol) was added dropwise. The mixture was stirred at RT for 20 min. (2S,3R,4R,5R)-2-acetoxy-5-((benzoyloxy)methyl)tetrahydrofuran-3,4-diyl dibenzoate (46.9 g, 93.0 mmol) was added in one portion, followed by dropwise addition of trimethylsilyl trifluoromethanesulfonate (15.2 mL, 78.7 mmol). The reaction mixture was stirred at 80° C. for 2 h. The reaction mixture was allowed to cool to RT and diluted with EtOAc (200 mL). The organic phase was washed with aq. satd. NaHCO₃ (100 mL) and brine (100 mL), dried over anhydrous Na₂SO₄, filtered, concentrated in vacuo, absorbed onto Celite and purified by flash column chromatography (Hept:EtOAc 90:10 to 70:30, KP-Sil 330 g) to yield 3 (31.0 g, 60%) as a white solid.

¹H NMR (500 MHz, CDCl₃) δ 8.58 (s, 1H), 8.09-8.13 (m, 2H), 7.99 (dd, J=8.3, 1.2 Hz, 2H), 7.92 (dd, J=8.4, 1.2 Hz, 2H), 7.49-7.64 (m, 6H), 7.39 (dt, J=22.8, 7.8, 7.8 Hz, 4H), 6.67 (d, J=5.4 Hz, 1H), 6.09-6.17 (m, 2H), 4.90 (dd, J=12.3, 3.1 Hz, 1H), 4.80 (q, J=3.5, 3.5, 3.4 Hz, 1H), 4.68 (dd, J=12.3, 3.6 Hz, 1H). ¹³C NMR (125 MHz, CDCl₃) δ 166.2, 165.5, 165.2, 153.3, 151.4, 151.1, 133.94, 133.91, 133.7, 132.1, 129.98, 129.96, 129.8, 129.4, 128.9, 128.8, 128.71, 128.66, 128.5, 117.9, 86.9, 80.8, 74.3, 71.6, 63.6, 53.8. HRMS (ESI-TOF) m/z calcd. for C₃₂H₂₃ClIN₃O₇ [M+H]+: 724.0347, found: 724.0384.

(2R,3R,4R,5R)-2-((Benzoyloxy)methyl)-5-(4-chloro-5-(4,4,5,5-tetramethyl-1,3,2-dioxaborolan-2-yl)-7H-pyrrolo[2,3-d]pyrimidin-7-yl)tetrahydrofuran-3,4-diyl dibenzoate 10

Compound 9 (31 g, 42.8 mmol) and tetrakis(triphenylphosphine)palladium(0) (0.99 g, 0.86 mmol) were dissolved in THF (360 mL) to which triethylamine (59 mL, 428 mmol) was added. The reaction mixture was cooled to −78° C. and stirred for 5 min before 4,4,5,5-tetramethyl-1,3,2-dioxaborolane (9.3 mL, 64.2 mmol) was added dropwise to the mixture. The reaction mixture was then allowed to warm to RT and successively heated to 80° C. for 36 h. The reaction mixture was allowed to cool to RT, concentrated in vacuo, absorbed onto Celite and purified by flash-chromatography (KP-Sil, 330 g, Hept: EtOAc, 95:5 to 70:30 to yield the target compound (10, 23.5 g, 76%) as a white solid.

¹H NMR (500 MHz, DMSO) δ 8.58 (s, 1H), 8.33 (s, 1H), 7.98 (ddd, J=11.8, 8.3, 1.2 Hz, 4H), 7.83 (dd, J=8.3, 1.2 Hz, 2H), 7.59-7.69 (m, 3H), 7.50 (dt, J=9.8, 8.1, 8.1 Hz, 4H), 7.39-7.43 (m, 2H), 6.76 (d, J=5.4 Hz, 1H), 6.35-6.39 (m, 1H), 6.14-6.18 (m, 1H), 4.79-4.89 (m, 2H), 4.69 (dd, J=12.1, 5.0 Hz, 1H), 1.28 (s, 12H). ¹³C NMR (126 MHz, DMSO) δ 165.4, 164.7, 164.4, 152.8, 152.5, 150.9, 138.7, 133.94, 133.87, 133.5, 129.4, 129.3, 129.23, 129.17, 128.75, 128.72, 128.71, 128.6, 128.2, 119.8, 86.7, 83.6, 79.3, 73.3, 70.8, 63.5, 54.9, 24.5. HRMS (ESI-TOF) m/z calcd. for C₃₈H₃₅BClN₃O₉ [M+H]+: 724.2233, found: 724.2245.

tert-Butyl (3-lodonaphthalen-2-yl)carbamate 11

An oven-dried 3-necked 1L round-bottom flask equipped with a magnetic stir bar was charged with 3-iodo-2-naphthoic acid (24.8 g, 83.2 mmol) dissolved in toluene (320 mL) and triethylamine (14 mL, 100.0 mmol) was added. The flask was fitted with a reflux condenser and an addition funnel. The reaction was heated to reflux and diphenyl phosphorazidate (21.6 mL, 100.0 mmol) in toluene (80 mL) was added dropwise to the reaction mixture over a total period of 60 min. The rate of addition was kept to 1 drop every 2-5 s and bubbles was observed after 5 min of addition. The addition funnel was rinsed with additional toluene (20 mL). After 15 min of stirring at reflux the bubble formation stopped. After an additional 15 min the addition funnel was charged with 2-methylpropan-2-ol (40 mL, 416.0 mmol) in toluene (60 mL), which was cautiously (note: the formed intermediate is extremely reactive and must be handled with care) added dropwise to the reaction mixture at reflux. The reaction was stirred at reflux for an additional 3 h. The reaction mixture was allowed to cool to RT, transferred to a separatory funnel and the material was washed sequentially with water (3×500 mL), aq. satd. NaHCO₃ (3×250 mL) followed by brine (1×500 mL). The resulting orange solution was dried over MgSO₄, filtered and concentrated in vacuo to yield 5 (20.0 g, 65%) as a beige solid.

¹H NMR (500 MHz, DMSO) δ 8.63 (s, 1H), 8.52 (s, 1H), 7.94 (s, 1H), 7.86 (dd, J=14.9, 8.1 Hz, 2H), 7.46-7.54 (m, 2H), 1.48 (s, 9H). ¹³C NMR (126 MHz, DMSO) δ 153.4, 138.1, 136.1, 132.7, 132.2, 127.3, 126.8, 126.4, 126.1, 123.5, 96.7, 79.2, 28.1. HRMS (ESI-TOF) m/z calcd. for C₁₅H₁₆INO₂ [M+H]+: 370.0304, found: 370.0295.

(2R,3R,4R,5R)-2-((Benzoyloxy)methyl)-5-(5-(3-((tert-butoxycarbonyl)amino)naphthalen-2-yl)-4-chloro-7H-pyrrolo[2,3-d]pyrimidin-7- yl)tetrahydrofuran-3,4-diyl dibenzoate 12

Compound 10 (22 g, 30.4 mmol), tert-butyl (3-iodonaphthalen-2-yl)carbamate 11 (10.7 g, 29.0 mmol), bis(triphenylphosphine)palladium(II) dichloride (1.02 g, 1.45 mmol) and potassium carbonate (10.0 g, 72.4 mmol) were dissolved in DME (300 ml) and the reaction was stirred at 80° C. for 24 h. The reaction mixture was concentrated in vacuo, absorbed on Celite and purified by flash-chromatography (KP-Sil 330 g, Hept:EtOAc 90:10 to 40:60) to yield 6 (18.2 g, 75%) as a yellow solid.

¹H NMR (500 MHz, DMSO) δ 8.64 (s, 1H), 8.40 (s, 1H), 8.15 (s, 1H), 8.10 (s, 1H), 7.99 (d, J=7.3 Hz, 2H), 7.88-7.94 (m, 5H), 7.84 (d, J=6.6 Hz, 2H), 7.63-7.68 (m, 2H), 7.57 (t, J=7.3, 7.3 Hz, 1H), 7.4-7.53 (m, 8H), 6.87 (d, J=4.7 Hz, 1H), 6.39 (t, J=5.1, 5.1 Hz, 1H), 6.23 (t, J=5.9, 5.9 Hz, 1H), 4.91 (q, J=5.1, 5.0, 5.0 Hz, 1H), 4.82 (dd, J=12.1, 3.5 Hz, 1H), 4.71 (dd, J=12.1, 5.0 Hz, 1H), 1.27 (s, 9H). ¹³C NMR (125 MHz, DMSO) δ 165.4, 164.6, 164.5, 153.2, 151.5, 151.3, 150.5, 135.6, 134.0, 133.9, 133.5, 133.1, 130.6, 129.5, 129.4, 129.3, 129.2, 129.17, 128.9, 128.8, 128.7, 128.6, 128.5, 128.3, 127.4, 127.0, 126.5, 125.5, 125.1, 116.6, 112.9, 86.6, 78.8, 73.8, 31.2, 27.9, 22.1, 13.9. HRMS (ESI-TOF) m/z calcd. for C₄₇H₃₉ClN₄O₉ [M+H]+: 839.2484, found: 839.2485.

(2R,3R,4R,5R)-2-((Benzoyloxy)methyl)-5-(5-(3-((tert-butoxycarbonyl)amino)naphthalen-2-yl)-4-chloro-7H-pyrrolo[2,3-d]pyrimidin-7- yl)tetrahydrofuran-3,4-diyl dibenzoate 13

Compound 10 (22 g, 30.4 mmol), tert-butyl (3-iodonaphthalen-2-yl)carbamate 11 (10.7 g, 29.0 mmol), bis(triphenylphosphine)palladium(II) dichloride (1.02 g, 1.45 mmol) and potassium carbonate (10.0 g, 72.4 mmol) were dissolved in DME (300 ml) and the reaction was stirred at 80° C. for 24 h. The reaction mixture was concentrated in vacuo, absorbed on Celite and purified by flash-chromatography (KP-Sil 330 g, Hept: EtOAc 90:10 to 40:60) to yield 6 (18.2 g, 75%) as a yellow solid.

¹H NMR (500 MHz, DMSO) δ 8.64 (s, 1H), 8.40 (s, 1H), 8.15 (s, 1H), 8.10 (s, 1H), 7.99 (d, J=7.3 Hz, 2H), 7.88-7.94 (m, 5H), 7.84 (d, J=6.6 Hz, 2H), 7.63-7.68 (m, 2H), 7.57 (t, J=7.3, 7.3 Hz, 1H), 7.4-7.53 (m, 8H), 6.87 (d, J=4.7 Hz, 1H), 6.39 (t, J=5.1, 5.1 Hz, 1H), 6.23 (t, J=5.9, 5.9 Hz, 1H), 4.91 (q, J=5.1, 5.0, 5.0 Hz, 1H), 4.82 (dd, J=12.1, 3.5 Hz, 1H), 4.71 (dd, J=12.1, 5.0 Hz, 1H), 1.27 (s, 9H). ¹³C NMR (125 MHZ, DMSO) δ 165.4, 164.6, 164.5, 153.2, 151.5, 151.3, 150.5, 135.6, 134.0, 133.9, 133.5, 133.1, 130.6, 129.5, 129.4, 129.3, 129.2, 129.17, 128.9, 128.8, 128.7, 128.6, 128.5, 128.3, 127.4, 127.0, 126.5, 125.5, 125.1, 116.6, 112.9, 86.6, 78.8, 73.8, 31.2, 27.9, 22.1, 13.9. HRMS (ESI-TOF) m/z calcd for C₄₇H₃₉ClN₄O₉ [M+H]+: 839.2484, found: 839.2485.

(2R,3R,4S,5R)-2-(2,3,5,6-Tetraazacyclopenta[de]tetracen-2(6H)-yl)-5-(hydroxymethyl)tetrahydrofuran-3,4-diol 14

Compound 6 (7 g, 8.3 mmol) was dissolved in DMF (18 mL) to which 1,4-diazabicyclo[2.2.2]octane (7.5 mL, 16.7 mmol) and 2,3,4,6,7,8,9,10-octahydropyrimido[1,2-a]azepine (2.5 mL, 16.7 mmol) was added. The reaction mixture was stirred at 75° C. for 17 h. The reaction mixture was allowed to cool to RT and was subsequently co-evaporated with toluene (5×15 mL). The crude product was purified by flash chromatography (KP-Sil 100 g, DCM:MeOH 100:0 to 95:5) which yielded a mixture of products consisting of Boc- and de-Boc protected product (7a and 7b, 3.1 g). The material obtained was used in the next step without further purification. The mixture of compound 7a and 7b (2.85 g) was dissolved in MeCN (32 mL) and sodium methanolate (3.9 mL, 21.3 mmol) was added. The reaction mixture was stirred at RT for 1 h. The reaction mixture was concentrated in vacuo, absorbed onto Celite and purified by flash chromatography (KP-Sil 25 g, DCM:MeOH 100:0 to 90:10) to yield 8 (0.55 g, 17% over two steps) as a white solid.

¹H NMR (500 MHz, DMSO) δ 10.90 (s, 1H), 8.19 (s, 1H), 8.15 (s, 1H), 7.80 (d, J=7.8 Hz, 1H), 7.72 (d, J=8.1 Hz, 1H), 7.70 (s, 1H), 7.55 (s, 1H), 7.33-7.43 (m, 2H), 5.95 (d, J=6.4 Hz, 1H), 5.40 (d, J=6.5 Hz, 1H), 5.18 (d, J=4.5 Hz, 1H), 4.55 (q, J=6.3, 6.3, 6.3 Hz, 1H), 4.15 (q, J=4.6, 4.6, 4.6 Hz, 1H), 3.97 (q, J=3.8, 3.8, 3.8 Hz, 1H), 3.68 (d, J=12.0 Hz, 1H), 3.55-3.61 (m, 1H), 2.54 (s, 1H). ¹³C NMR (126 MHz, DMSO) δ 155.2, 153.6, 147.2, 137.4, 132.6, 129.6, 129.2, 128.5, 127.2, 126.6, 126.3, 124.7, 122.4, 120.3, 113.4, 113.3, 110.4, 106.7, 88.5, 85.6, 74.1, 70.9, 61.9. HRMS (ESI-TOF) m/z calcd. for C₂₁H₁₈N₄O₄ [M+H]+: 391.1406, found: 391.1405.

(2R,3R,4S,5R)-2-(2,3,5,6-tetraazacyclopenta[de]tetracen-2(6H)-yl)-5-((bis(4 methoxyphenyl)(phenyl)methoxy)methyl)tetrahydrofuran-3,4-diol 15

(2R,3R,4S,5R)-2-(2,3,5,6-Tetraazacyclopenta[de]tetracen-2(6H)-yl)-5-(hydroxymethyl)tetrahydrofuran-3,4-diol (150 mg, 0.38 mmol) was co-evaporated with pyridine (25 mL) thrice before pyridine (3 mL) was added, and the flask was placed in an ice bath (0° C.). DMTr-Cl (143 mg, 0.42 mmol) was then added in one portion and the reaction mixture was stirred for 5 min before being returned to RT and stirred for 18 h. Methanol (3 mL) was subsequently added and the reaction mixture was extracted with EtOAc (50 mL), washed with water (20 mL) and brine (10 mL). The organic layers were dried over MgSO₄, concentrated to dryness, adsorbed onto Celite and purified by flash chromatography (KP-Sil 25 g, flushed with 2% Et₃N in DCM prior to use, EtOAc in DCM: 0 to 50%) to yield compound 9 (60 mg, 23%) as a light brown solid.

¹H NMR (500 MHz, DMSO-d6) δ 10.90 (bs, 1H), 8.19 (s, 1H), 7.74 (s, 1H), 7.71 (d, J=8.0 Hz, 1H), 7.66 (d, J=9.9 Hz, 2H), 7.52 (s, 1H), 7.40 (d, J=7.5 Hz, 3H), 7.33-7.39 (m, 2H), 7.28 (t, J=8.3 Hz, 6H), 7.20 (t, J=7.3 Hz, 1H), 6.84 (dd, J=8.9, 6.9 Hz, 4H), 6.03 (d, J=4.5 Hz, 1H), 5.57 (bs, 1H), 4.67 (t, J=4.8 Hz, 1H), 4.39 (t, J=5.0 Hz, 1H), 4.07 (q, J=4.4 Hz, 1H), 3.66 (s, 3H), 3.65 (s, 3H), 3.22 (m, 2H). ¹³C NMR (126 MHz, DMSO-d6) δ 158.1, 158.0, 155.5, 153.6, 147.6, 144.8, 137.6, 135.7, 135.6, 132.7, 129.8, 129.7, 129.5, 127.9, 127.8, 127.1, 126.7, 126.6, 126.4, 124.7, 122.2, 120.3, 113.3, 113.2, 112.8, 110.7, 106.5, 88.4, 85.6, 82.8, 74.2, 70.6, 63.7, 55.0, 54.9. HRMS (ESI-TOF) m/z calcd. for C₄₂H₃₆N₄O₆ [M+H]+: 693.2713, found: 693.2729.

CPG-supported (2R,3R,4S,5R)-2-(2,3,5,6-tetraazacyclopenta[de]tetracen-2(6H)-yl)-5-((bis(4-methoxyphenyl)(phenyl)methoxy)methyl)tetrahydrofuran-3,4-diol 16

In a 10 ml syringe with PTFE filter, succinylated support 3 (400 mg, 82 μmol/g, 0.03 mmol), DMAP (0.008 g, 0.07 mmol), DIC (203 μl, 1.31 mmol) and (2R,3R,4S,5R)-2-(2,3,5,6-tetraazacyclopenta[de]tetracen-2(6H)-yl)-5-((bis(4-methoxyphenyl)(phenyl)methoxy)methyl)tetrahydrofuran-3,4-diol (0.023 g, 0.03 mmol) and triethylamine (14 μl, 0.10 mmol) were suspended in pyridine (5 mL). The mixture was gently shaken for 18 h at RT. After 18 h, the syringe was purged and the support washed with pyridine (5 mL), dichloromethane (5 mL) and diethyl ether. A second coupling was then performed in the exact same conditions as the first to increase loading on the solid support. Subsequently, in the same syringe, DMAP (0.008 g, 0.07 mmol), DIC (203 μl, 1.31 mmol), triethylamine (14 μl, 0.10 mmol) and 2,3,4,5,6-pentachlorophenol (0.087 g, 0.33 mmol) were added to the support and suspended in pyridine (4 mL). The mixture was gently shaken for 4 h at RT before a solution of piperidine (4 mL, 20% in DMF—for capping of the unreacted carboxylic acids on the support) was added for 1 min (longer exposure time will reduce loading as piperidine cleaves the ester bonds with the nucleoside), then quickly washed away with DMF (3'5 mL), dichloromethane (5 mL) and diethyl ether (5 mL). Finally, the resin was shaken in a CAP A+CAP B mix (50/50 v/v) for 2 hours under argon atmosphere, then washed with DMF (5 mL), dichloromethane (5 mL), diethyl ether (5 mL) and argon-dried (final loading: 18 μmol/g−determined by reading optical density of a DMT solution cleaved from a weighed amount of support−ε=70000 M⁻¹·cm⁻¹ at 498 nm).

((2R,3S,4R,5R)-5-(2,3,5,6-tetraazacyclopenta[de]tetracen-2(6H)-yl)-3,4-dihydroxytetrahydrofuran-2-yl)methyl hydrogen triphosphate (pATP) 17

Reaction was performed in a 5 ml syringe with PTFE filter loaded with the CPG-bound pA nucleoside (400 mg, 0.007 mmol) under an argon atmosphere and shaking. Steps were performed as following:
a. 5′-DMT removal: the support was washed with a flow of DCA deblock until the filtrate was colourless, then washed with ACN (5×5 mL).
b. Coupling: N,N-diisopropyl-4H-benzo[d][1,3,2]dioxaphosphinin-2-amine 5 (345 mg, 1.36 mmol) was dissolved in 4.8 mL ACN and reacted portionwise with the support (3 equal couplings with reaction times 60 s-60 s-90 s respectively). To each coupling, BTT activator (2.4 mL) was also added. The support was subsequently washed with ACN (3×5 mL).
c. Oxidation: Pyridine/Water/lodine (9/1/12.7 v/v/w, 5 mL) for 45 s, followed by ACN wash (3×5 mL) and drying of the support in an argon flow.
d. Triphosphorylation: Two injections of bis(tetrabutylammonium) dihydrogen diphosphate 6 (0.5 M, 5 ml) for 15 min and 18 hours, respectively. The support was subsequently rinsed with DMF (5 mL), water (3×5 mL), ACN (5 mL) and then dried in an argon flow.
e. Cleavage and Purification: Cleavage of the triphosphate was done in 2 h at room temperature with AMA (50/50 v/v mix of 23% aq. NH₄OH and 40% aq. methylamine, 5 mL). After 2 hours, the AMA filtrate was purged in a round-bottom flask and the support was rinsed 3 times with 23% aq. NH₄OH solution. After freeze-drying of the mixture, purification by HPLC (Waters Acquity HSS T3 column, 2.1×50 mm, 0.4 mL/min, 2 to 99% 50 mM NH₄OAc in water 80:20 EtOH) was performed to furnish pATP (2.7 mg, 60% determined from UV absorbance) as a light-yellow solid (ammonium salt).

³¹P NMR (202 MHz, D2O) δ −22.88 (t, J=17.7 Hz, 1P), −11.14 (d, J=19.7 Hz, 1P), −10.21 (bs, 1P). HRMS (ESI-TOF) m/z calcd. for C₂₁H₂₁N₄O₁₃P₃ [M+H]+: 631.0396, found: 631.0416.

pATP 17 can also be made by a slightly modified route wherein the coupling step (b above) is carried out with a modified phosphoramidite such as 6-chloro-N,N-diisopropyl-4-methyl-4H-benzo[d][1,3,2]dioxaphosphinin-2-amine 8 (compound (IIIa) above).

### Example 3: Photophysical Characterisation

Absorption spectra were recorded on a Cary 4000 spectrometer (Agilent Technologies). The emission spectra were measured on a SPEX Fluorolog 3 spectrofluorimeter (Jobin Yvon Horiba). Three consecutive emission spectra recorded at a scan rate of 600 nm min−1 were collected and averaged. The spectra of compounds 7 and 17 in DPBS were recorded from 360 nm to 695 nm with excitation at 355 nm. To avoid bleaching, the monochromator slits on the excitation side were set to 0.8 nm, and on the emission side to 2.5 nm.

The spectra of molar absorptivities (ε in M⁻¹cm⁻¹) were calculated using Equation 1, with A being the measured absorption, A260 and ε260 the absorption or molar absorptivity, respectively, at 260 nm.

$$ε ( λ ) = A ( λ ) * \frac{{ε}_{260}}{{A}_{260}} \qquad [ 1 ]$$

The fluorescence quantum yields were determined relative to the quantum yield of quinine sulphate in 0.5 M H₂SO₄ (ϕ_{F}=0.546⁴², referred to as reference), and calculated using Equation 2, in which subscripts s and r refer to sample and reference, respectively. I is the integrated fluorescence intensity, A the measured absorption of the fluorophore at the excitation wavelength and n is the refractive index of the solvent (H₂O or H₂SO₄, respectively; Eaton D. F.; Pure Appl. Chem. 60, 1107-1114 [1988 ]).

$${Φ}_{F , S} = \frac{{I}_{s} / {A}_{s}}{{I}_{r} / {A}_{r}} \frac{{n}_{s}^{2}}{{n}_{r}^{2}} * {Φ}_{F , r} \qquad [ 2 ]$$

The spectroscopic properties of compounds 7 and 17 were determined under physiological conditions in order to determine the experimental setup for cell studies, with absorption and emission spectra being recorded in DPBS to mimic the ionic strength and salt composition in mammalian cells. To record emission spectra, the excitation wavelength was set to 355 nm, and measurements were performed at room temperature (ca. 22° C.). Molar absorptivities (s) were then calculated using the Beer-Lambert law.

Apart from absorption where the common nucleobases absorb (<300 nm), compounds 7 and 17 absorb in the 350-400 nm range, with 2CNqATP displaying an absorption maximum at 352 nm (Table 2). The pATP absorption band on the other hand has two peaks, at 370 nm and 386 nm respectively, the latter being slightly more intense. The emission peaks are centred at 463 nm and 420 nm for 2CNqATP and pATP, respectively. FBA refers to “fluorescent base analogue”.

TABLE 1
Spectroscopic parameters of compounds
7 and 17 in DPBS at room temperature

| | | | | | ε_{max, FBA} | ε_{405, FBA} |
|---|---|---|---|---|---|---|
| | ε₂₆₀ | | | | χ Φ_{F} | χ Φ_{F} |
| | (M⁻¹ | Φ_{F} | λ_{abs, max} | λ_{em, max} | (M⁻¹ | (M⁻¹ |
| Compound | cm⁻¹) | (%) | (nm) | (nm) | cm⁻¹) | cm⁻¹) |
| 2CNqATP | 14600²⁶ | 47 | 352 | 463 | 4900 | 504 |
| pATP | 22300²⁷ | 49 | 386 | 420 | 6000 | 909 |

### Example 4: Cell Culture

The Huh-7 human liver cell line was used. Huh-7 is a well-differentiated human hepatic cell line with epithelial-like morphology.⁴³ The cells were cultured at 37° C. under 5% CO₂ in Dulbecco's modified Eagle medium (DMEM GlutaMax with added phenol red, Gibco) containing 4.5 g/l glucose with an addition of 10% foetal bovine serum (FBS, Gibco, origin Brazil), 2 mM L-Glutamine, and 1 mM sodium pyruvate (hereafter referred to as CCM, complete cell culture medium). For sub-cultivation the adherent cells were washed twice with DPBS, containing no calcium or magnesium, and detached with 0.25% trypsin ethylenediaminetetraacetic acid 1x (trypsin-EDTA, Gibco, with phenol red). For seeding, the cells were counted after trypsin neutralization, diluted to the desired number of cells (Table 2) and thereafter incubated at 37° C. with 5% CO₂ for 24 h before experiments.

TABLE 2
Reservoirs used for the seeding of Huh-7 cells
with indicated cell number and working volume

| | Number of cells | Working volume | |
|---|---|---|---|
| Vessel | per well | per well | |
| 96 well plate | 18000 | 100 | μL |
| 48 well plate | 45000 | 250 | μL |
| 12 well plate | 106 | 1 | mL |
| 4 chamber dish with glass bottom | 45000 | 250 | μL |

(Greiner Bio-One)

### Example 5: Cytotoxicity

To investigate the cytotoxicity of compounds 7 and 17 Huh-7 cells were seeded in 96-well plates as described above. Prior to treatment, the conditioned medium was removed from the Huh-7 cells, and the compounds (stock solution in DPBS diluted in CCM) were added to the cells for different exposure times. Unexposed cells were treated the same way, with the same amount of added DPBS to CCM, instead of the compounds. Two different cytotoxicity assays (alamarBlue to measure metabolic activity and LDH leakage to measure cell membrane integrity) were performed in parallel using the cells and the culture medium from each sample, respectively. Treatments were done in triplicates and the experiment was repeated twice.

After 24 h exposure with compounds 7 or 17, the conditioned medium was removed from the cells and used for the LDH leakage assay (see below). Freshly prepared alamarBlue cell viability reagent (Invitrogen, diluted 1:10 in CCM) was added, followed by incubation for 3 h at 37° C. and 5% CO₂. The resulting resorufin fluorescence was read using an Optima Fluostar plate reader (BMG Labtech) Ex: 544 nm/Em: 590 nm. Dimethyl sulfoxide 5% and CCM alone (i.e. compounds 7 and 17 0 μM) were used as positive and negative controls for cell death, respectively.

Lactate Dehydrogenase (LDH) Leakage Assay: To test for released LDH, the CyQUANT LDH Cytotoxicity Assay kit (Invitrogen) was used. Reaction mixes and enzymatic control (LDH, pure enzyme) were prepared according to the manufacturer's instructions. Maximum LDH release were determined by treating cells with a 1:10 dilution of lysis buffer in serum free medium for 30 min at 37° C. and 5% CO₂.

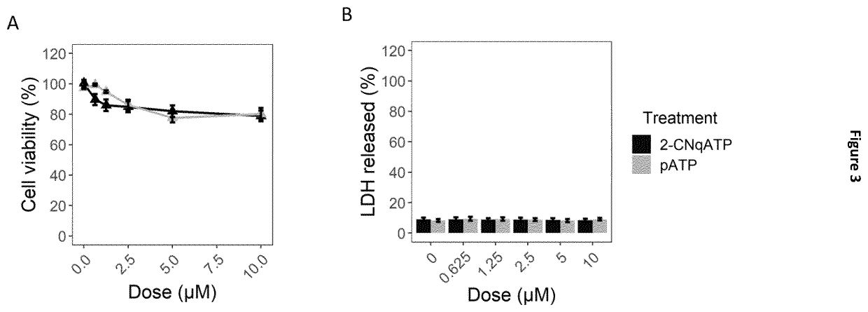

Results: The alamarBlue assay reports metabolic activity of cells and, hence, informs about cell viability. The LDH leakage assay reports on the membrane integrity of the cells, constituting a complementary read-out of cytotoxicity (membrane damage). FIG. 3 shows that 2CNqATP and pATP induced maximally 20% of cell death at the two highest tested concentrations of 10 μM (FIG. 3A). Comparison of cell viability at lower doses suggests that 2CNqATP is slightly more toxic than pATP. The level of released LDH was about 10% for all exposure conditions and in parity with the value obtained for untreated control cells, demonstrating that cell integrity is retained. Based on the results, cell uptake experiments were conducted at a concentration of 2.5 μM, which gave reliable intracellular fluorescence signals and in-cellulo RNA incorporation, with low effects on cell viability following 24 h exposure.

As a further observation on viability, no morphological changes or other indications of cell death (such as detachment from the vessel surface) were found during live-cell imaging. This shows that the excitation light used for tracking compounds in living cells does not harm the cells under the tested conditions.

### Example 6: Monitoring Cellular Uptake Using Fluorescence Microscopy

Cellular uptake screening for compounds 7 and 17 was performed using the following methods.

Quantification in Cell Lysate: For dose response experiments of the uptake of compounds 7 and 17, total cell lysates of the exposed cells were collected and measured on an Optima Blue Fluostar plate reader (BMG Labtech) with lysis buffer as background. 2CNqATP was excited using 355 nm (±20 nm) filter and emission was collected at through a bandpass filter centred at 460 nm (±12 nm). pATP was excited using 380 nm (±5 nm) filter and emission bandpass filter centred at 410 nm (±5 nm). Lysate buffer fluorescence was subtracted as background, and the fluorescence intensities of the triplicates were averaged. The fluorescence intensity was normalized to the highest added concentration of the compounds (10 μM).

Confocal Laser Scanning Fluorescence Microscopy: Images were captured using a Nikon eclipse Ti microscope with a Nikon C2plus scanner, two parallel Gallium arsenide phosphide (GaAsP) detectors, and an Apo 60x Oil μS DIC N2 objective. To excite the fluorescent nucleoside triphosphates the 405 nm laser line was used, and the emission was collected between 407-607 nm. To optimize the signal while avoiding photobleaching of the compounds a laser power of 2% and a detector gain of 4% was applied. For live cell imaging, Huh-7 cells were seeded in a four-compartment dish (CELLview Dishes, Greiner Bio-One) with glass bottom as described above. During imaging the dish was placed in a stage top incubation chamber (OKO lab) maintaining 37° C. and 5% CO₂.

Time-lapse Imaging: To image the uptake of compounds 7 and 17 over time, the time-lapse setup of the NIS software was used. For every compartment of the dish, one field of view was chosen, from which images were captured every 15 minutes in the first 2 hours, and then every hour over 18 hours (i.e. total time of 20 hours). Exposure time started by adding pre-warmed (37° C.) CCM containing 2.5 μM of FBA-TP to the cells. The fresh CCM for the control cells contained an equal volume of DPBS instead of compound solution.

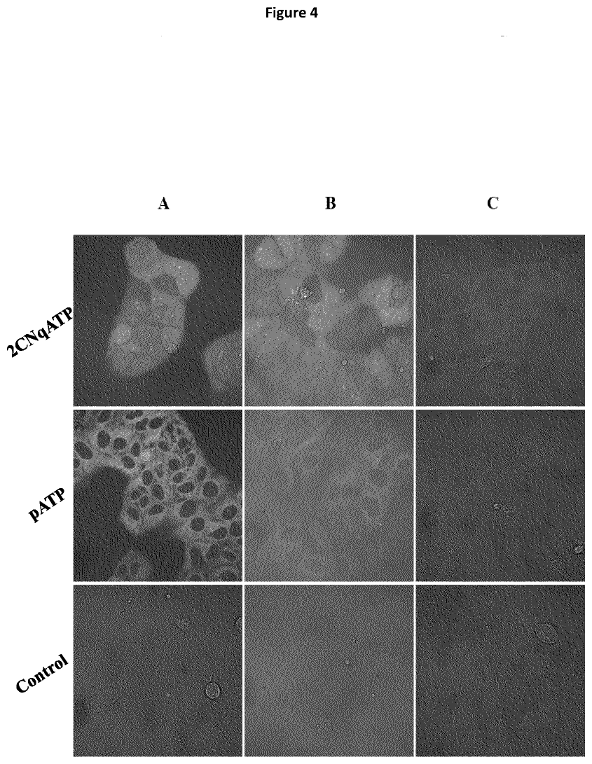

Results: To explore the cellular uptake of compounds 7 and 17, they were added to Huh-7 cells in CCM and the uptake was monitored as function of time using a confocal fluorescence microscope (FIG. 4). Control cells, exposed to CCM alone, were imaged using the same microscopy settings and recorded as reference to probe for cellular autofluorescence levels. After 20 h of continuous uptake, clear differences in the accumulation and localisation of the compounds were observed.

2CNqATP (compound 7) is seemingly evenly distributed across the cytosol and cell nuclei. The bright spots inside of individual nuclei, further indicate an accumulation also in nucleoli. By contrast, pATP accumulates in the cytosol, but not in the cell nucleus. Moreover, its distribution in the cytoplasm is not even, instead it appears to localise with some preference to intracellular structures near the nuclei, which could be part of the endoplasmic reticulum or Golgi.

No uptake of the compounds was observed when the Huh-7 cells were kept at 4° C. indicating that the modified triphosphates enter cells via an energy-dependent mechanism and not via passive diffusion across the plasma membrane.

### Example 7: Uptake Kinetics Monitored by Flow Cytometry

For flow cytometry experiments, cells were seeded in a 48-well plate as described above. For kinetics experiments of the uptake, solutions of 2.5 μM of compounds 7 or 17 in CCM were added. Control samples were CCM, containing an equal volume of DPBS instead of FBA-TP. All treatments were done in triplicate. The experiment was setup such that all samples could be harvested at the same time at the end of the experiment. The FBA-TP solutions where therefore added at different time points. At the end of the experiment, the cells were washed twice with DPBS, and thereafter harvested by addition of 0.25% trypsin-EDTA. The trypsin was neutralized by adding DPBS with 2% added FBS and samples were transferred to a 96 U-bottom well plate for high throughput readout.

Measurements were generally performed on a BD LSRFortessa flow cytometer with an BD High Throughput Sampler (HTS). The system was connected to the software BD FACSDiva. Samples were excited using a 405 nm laser and emission passed through a bandpass filter centred at 450 nm (±20 nm). Uptake kinetics experiments were repeated twice. For monophosphate compounds, a Luminex CellStream flow cytometer with a high throughput sampler (HTS) connected to the CellStream Acquisition software was used. Excitation wavelength was 405 nm, with the emission passing through a 456/51 nm bandpass filter.

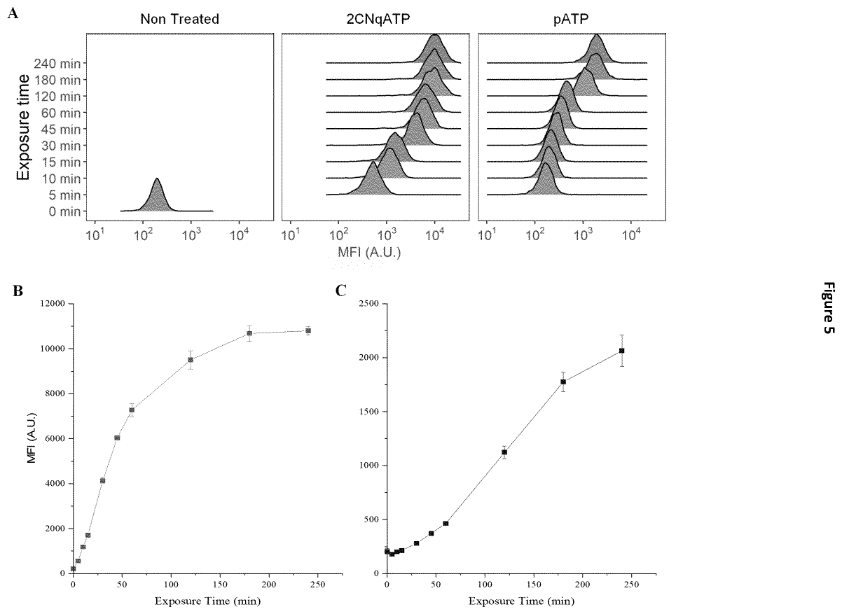

Results: Cells were exposed to compounds 7 and 17 for set times, ranging from 5 min to 4 h. Prior to flow cytometry analysis, the cells were washed and trypsinized to remove any potential extracellular compounds 7 and 17. For each time point and treatment condition, the intracellular fluorescence intensity of individual cells was recorded (FIG. 5A). The intensity histograms show a continuous increase in cellular fluorescence for both compounds, albeit significantly slower for pATP compared to 2CNqATP. The kinetic traces of the uptake (FIG. 5 B, C) reveal that 2CNqATP fluorescence is significantly over the initial (t=0) level already after 5 min, where after the uptake increases rapidly, reaching a plateau after 2 h exposure time (FIG. 5 B). The pATP uptake displays a lag phase during the initial 15 min and is then internalised at a near constant rate during the following 3 h (FIG. 5 C). The pATP uptake does not reach saturation within the 4 h experiment time frame. This finding clearly shows different uptake kinetics of compounds 7 and 17.

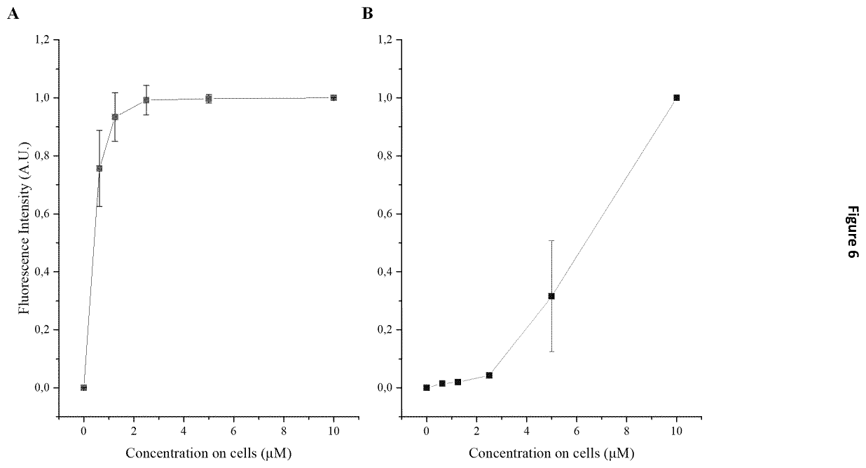

Dose-response experiments were performed at one selected time point (24 h) in order to investigate the concentration dependency of cellular FBA-TP uptake. At the end of exposure, the cells were washed and lysed. The fluorescence of the compounds in the lysate, reflecting the internalized FBA, was measured using a plate reader. For pATP and 2CNqATP, an increase in fluorescence emission in the lysate was observed with increasing concentration of added compound (FIG. 6). Compounds 7 and 17 show very different uptake characteristics. Cellular uptake of 2CNqATP increases dramatically with small increases in concentration and reaches a maximum, indicative of uptake saturation, already when 2.5 μM FBA-TP is added to the cells. By contrast, the increase of pATP emission between 0 and 2.5 μM is very small, only 4% of that of 2CNqATP, but when FBA-TP concentration is doubled it increases about 7 times. By adding 10 μM FBA-TP the fluorescence increases about 25 times, compared to 2.5 μM added compound.

### Example 8: Uptake Competition with Canonical Nucleoside Triphosphate and Nucleoside

Uptake of 2CNqATP and pATP was compared with natural adenosine triphosphate (ATP) or adenosine (both purchased from Sigma-Aldrich). The readout is based on the fluorescence of the compounds, detected in living cells by flow cytometry or in cell lysate using a microplate reader following co-administration with increasing concentrations of natural ATP or adenosine.

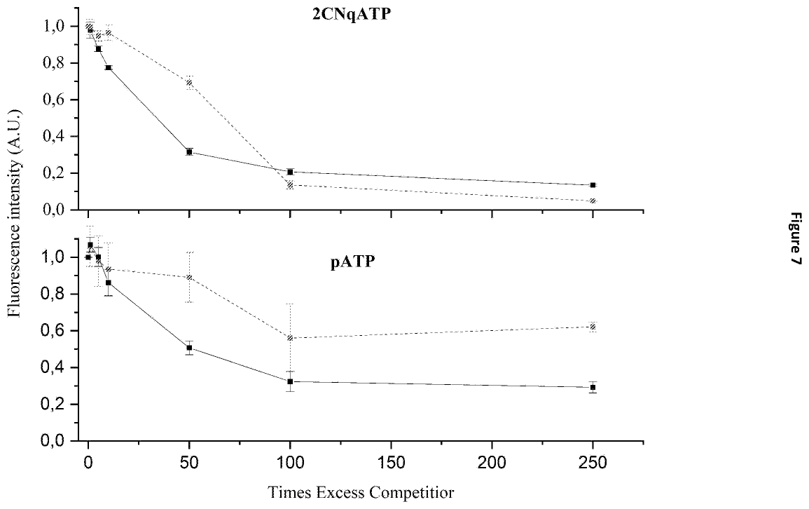

Cells were seeded in a 96-well plate. Treatment solutions with ATP or adenosine were prepared by stepwise dilution in CCM to reach the concentrations shown in FIG. 7. 2CNqATP or pATP were added at a concentration of 2.5 μM. The cells were incubated for 4 h at 37° C. and 5% CO₂. Cell morphology was checked under a light microscope.

For flow cytometry, the treated cells were washed three times with DPBS and detached with 0.25% trypsin-EDTA, which was then neutralized with CCM. Samples were transferred to a 96 U-well plate for high throughput readout on a BD LSRFortessa flow cytometer. Samples were excited using a 405 nm laser and emission passed through a bandpass filter centred at 450 nm (±20 nm).

For lysate readout, cells were lysed in 5x passive lysis buffer (Promega) for 1 h rocking at room temperature (ca. 22° C.). Fluorescence was recorded on an Optima Blue Fluostar plate reader (BMG Labtech). 2CNqATP was excited at 355 nm (±20 nm) and emission was detected using bandpass filter centered at 460 nm (±12 nm). For pATP the excitation was placed at 380 nm (±5 nm) and emission centered at 410 nm (±5 nm). The mean emission of duplicate sample was averaged and normalized to the fluorescence intensity of the cells without added competitor (ATP or adenosine).

Results: The results are shown in FIG. 7. The cellular (or lysate) fluorescence of the compounds is decreased with increasing (super-stoichiometric) additions ATP or adenosine as competitors, but the response is different. 2CNqATP competes with both ATP and adenosine with a stronger competition effect for ATP at low concentrations. The uptake of pATP also competes with ATP and adenosine, but the effect is considerably weaker.

The results suggest that the compounds, in part, share uptake pathways with canonical ATP and adenosine.

### Example 9: Cellular Incorporation Into RNA

RNA Extraction: Cells were seeded in 12-well plates as described above. Treatment solutions were prepared by diluting compounds 7 and 17 to a concentration of 2.5 μM or 5 μM in CCM. Conditioned medium was removed from the cells and treatment solutions were added. For control cells CCM alone was added. Cells were incubated for 24 h at 37° C. and 5% CO₂. For RNA extraction and purification, a QIAGEN RNeasy Mini Kit was used, following the manufacturer's protocol. Briefly, the cells were washed with DPBS, lysed, homogenized by 12 times passing it through a 20-gauge needle (0.9 mm outer diameter), and added to an equal volume of ethanol. The resulting solution was transferred to a RNeasy spin column and centrifuged. Then, binding buffer was added on top of the column, where after it was centrifuged again. The column-bound RNA was washed five times with washing buffer, with a centrifugation step between each addition. To elute the extracted RNA, 30 μL of the provided RNase-free water was applied to the column and centrifuged. This step was repeated twice to increase the yield of extracted RNA.

Three control samples were prepared to examine potential FBA-TP interferences with the extraction procedure, as well as non-covalent binding between the FBA-TP and RNA. 1) FBA-TP was added to the kit's lysate buffer to a concentration of 2.5 μM and treated in the same way starting from the homogenization step. 2) Unexposed cells were seeded and lysed as described above and FBA-TP was thereafter added directly to the lysate to make up a 2.5 μM final concentration. 3) FBA-TP added to pre-extracted RNA from unexposed cells was included. The concentration and purity of the RNA extracted from the unexposed cells were determined using a NanoDrop 2000 spectrophotometer (Thermo Scientific) and FBA-TP was added to reach a final concentration of 2.5 μM in the solution. All control samples were applied to RNeasy spin columns and treated the same way as the compound-exposed cell samples, following the purification protocol described above.

Absorption and Emission Readout of cellular RNA: Absorption spectra of extracted and purified RNA samples and controls were recorded on a Cary 4000 spectrometer (Agilent Technologies) between 200 nm and 600 nm. Emission spectra were measured on a SPEX Fluorolog 3 spectrofluorimeter (Jobin Yvon Horiba). 2CNqA was excited at 350 nm and pA was excited at 370 nm. The excitation and emission bandpass were 2 nm and 3 nm respectively. For excitation spectra the emission wavelength was set to 443 nm.

Results: The incorporation of the compounds into cell endogenous RNA was examined by harvesting RNA from exposed cells using a commercial kit for RNA extraction and purification. Control samples were prepared to exclude that non-incorporated compound interfered with the read-out (due to passive interactions with the purified RNA).

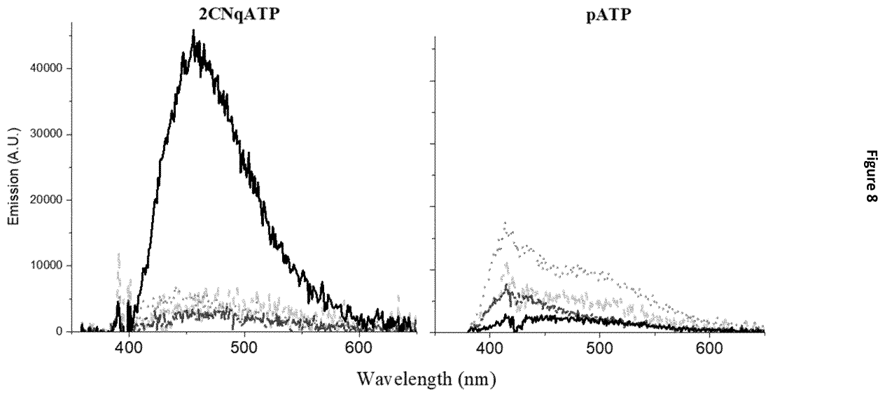

After purification, the emission and absorption spectra of the extracted cellular RNA were recorded (FIG. 8). The emission spectra of the compounds were normalized to the corresponding absorption value at 260 nm, to compensate for RNA concentration variations. All control samples display very low emission, showing that there is little to no non-covalent interaction between compounds 7 and 17 and RNA, or binding to the RNA purification column material. Furthermore, it shows that the purification method effectively separates RNA and free triphosphates.

The emission of the RNA samples of cells exposed to pATP are on the same level as the controls and do not show distinct peaks (FIG. 8, right graph). The RNA sample from cells exposed to 2CNqATP shows a significant emission peak with a maximum at 460 nm upon excitation at 350 nm (FIG. 8, left graph), demonstrating in-cellulo labelling of RNA.

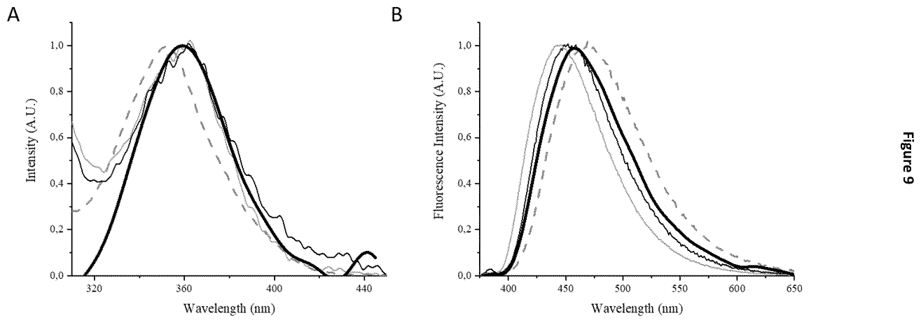

The results with 2CNqATP incorporation were verified by analysing spectral differences between RNA-incorporated 2CNqA and free 2CNqATP (FIG. 9). The excitation/absorption spectra (FIG. 9A) show a clear redshift of about 10 nm is observed for the extracted 2CNqA-RNA compared to free 2CNqATP (FIG. 9A; black thick line vs grey dashed line) but overlaps well with the absorption of single stranded RNA (ssRNA) and double stranded RNA (dsRNA) solid phase synthesized 25 mers containing 2CNqA (FIG. 9A; black and grey thin lines).

The emission spectra of extracted 2CNqA-RNA, free 2CNqATP and artificial RNA were also compared (FIG. 9B). The maximum emission of extracted 2CNqA-RNA (FIG. 9 B; black thick line) is about 20 nm blue shifted compared to the free 2CNqATP (FIG. 9 B; grey dashed line). The peaks of the synthetic short ssRNA and dsRNA are also blue shifted but to a greater extent than for the cell-extracted 2CNqA-RNA (FIG. 9 B; black thin line and grey thin line respectively). The cell-extracted 2CNqA-RNA emission agrees best with that of ssRNA indicating its likely single-stranded nature.

### Example 10: Monophosphate Experiments

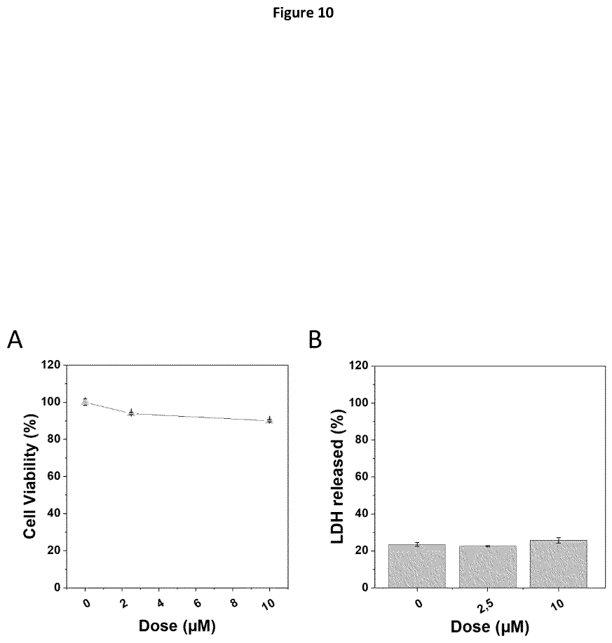

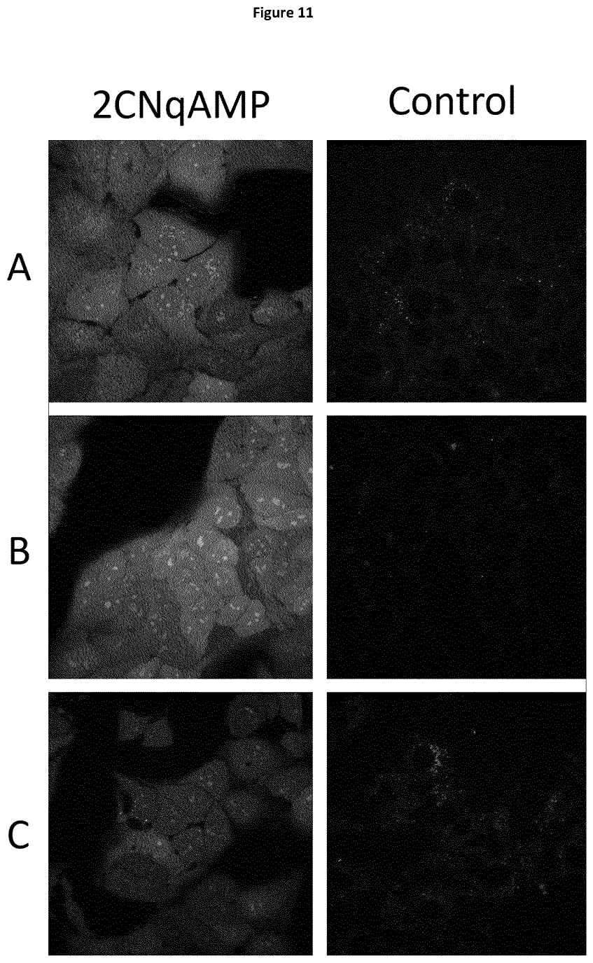

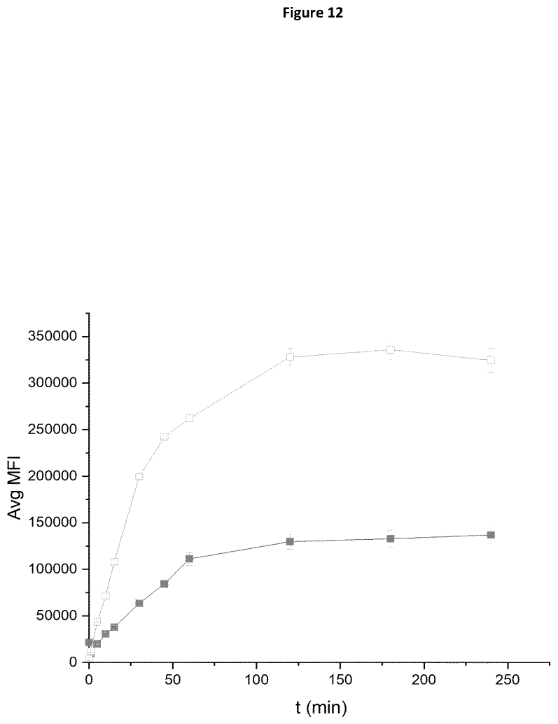

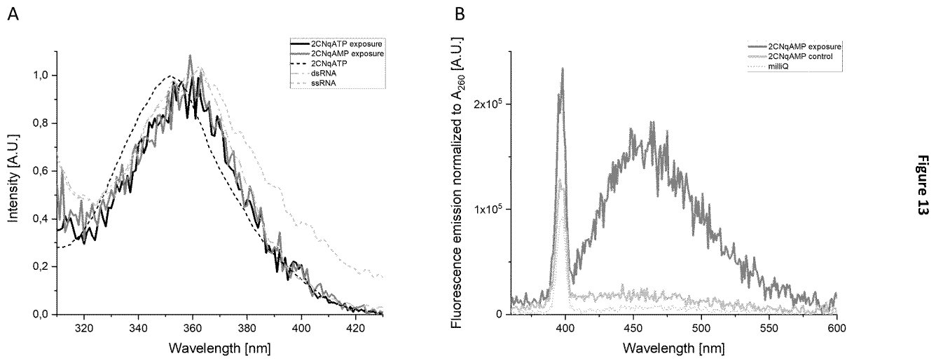

Analogous experiments to those described above were also carried out using compound 7a (2CNqAMP) as a substrate in place of compounds 7 and/or 17 (see FIGS. 10-13). These showed that the equivalent monophosphate compounds are also non-toxic and display similar properties to their triphosphate relations with respect to spontaneous cellular uptake and RNA incorporation.

### Conclusions

The compounds of formula (I) disclosed in this specification are shown to spontaneously internalise into live cells leading to in-cellulo incorporations into endogenous RNA. Cytotoxicity testing by alamarBlue assay showed that the compounds of formula (I) display about 20% reduction in cell metabolic activity at the highest measured concentration (10 μM), whereas the LDH assay disclosed no disruption of cell membrane integrity compared to untreated cells. This shows that the compounds, at the concentrations needed to easily image their locations within cells by confocal fluorescence microscopy and achieve in-cellulo RNA incorporation (2.5 μM), are well suited for experiments in a cellular context.

Both compounds 7 and 17 gave rise to strong intracellular fluorescence following 20 h exposure to Huh-7 cells. Lack of cell uptake at 4° C. demonstrated that internalisation proceeds via an active process, e.g. involving protein transporters, endocytosis or similar energy-dependent mechanisms and not by simple passive diffusion across the phospholipid bilayer. Uptake of the compounds is, in part, competed by natural ATP/adenosine Since confocal microscopy images (FIG. 4) show that the compounds 7 and 17 distribute evenly across the cytosol, it is not likely that the uptake is vesicle mediated. The results also show that the chemical modification of the FBAs compared to the canonical nucleotides enhances their uptake.

pATP and 2CNqATP accumulate in different intracellular locations. pATP accumulates preferentially in the cytosol (with some marked localisation at intracellular structures around the nuclei), while 2CNqATP is seemingly evenly distributed across the cytosol and nuclei. This means that 2CNqATP, beside passing the cellular membrane via an active mechanism, is also effectively retained in the nucleus with a particular accumulation in the nucleoli. Nucleoli are sites of ribosome biogenesis (ribosomal RNA transcription, formation, and maturation; see Hadjiolov, A. A. “The Nucleolus and Ribosome Biogenesis” vol. 12 (Springer Vienna, 1985) suggesting that 2CNqATP, when spontaneously incorporated into cell-synthesized RNA, could be used to fluorescently label ribosomal RNA.

Similar results were also obtained when using monophosphate compounds such as 7a (2CNqAMP) rather than their triphosphate analogues.

The results summarised above demonstrate that compounds of formula (I) are unexpectedly and spontaneously (without need for formulation or transfection reagents) internalised by suitable cells. Their uptake and processing characteristics are highly dependent on the structural modification of the base part of the molecule, such that the compounds have different intracellular localisation, uptake kinetics, dose response, and behaviour in competition with the corresponding canonical nucleosides. Given that it is possible to isolate RNA from treated cells comprising labels derived from the compounds of formula (I), this technology offers unique opportunities for novel fluorescence labelling of endogenous RNA in-cellulo.

1. A compound of formula (I), a physiologically cleavable precursor or a salt thereof: 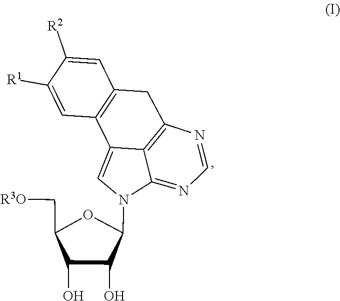
where R¹ is selected from hydro and R² is selected from cyano, or R¹ and R² together with the atoms to which they are attached form a 6-membered carboaromatic ring and R³ is selected from —P(O)(OH)₂, —P(O)(OH)—O—P(O)(OH)₂, and —P(O)(OH)—O—P(O)(OH)—O—P(O)(OH)₂.

2. The compound of formula (I), a physiologically cleavable precursor or a salt thereof as claimed in claim 1 where R¹ is hydro and R² is cyano.

3. The compound of formula (I), a physiologically cleavable precursor or a salt thereof as claimed in claim 1 where R¹ and R² together with the atoms to which they are attached form a 6-membered carboaromatic ring.

4. The compound of formula (I), a physiologically cleavable precursor or a salt thereof as claimed in claim 1 where R³ is selected from —P(O)(OH)₂ and —P(O)(OH)—O—P(O)(OH)—O—P(O)(OH)₂.

5. The compound of formula (I), a physiologically cleavable precursor or a salt thereof as claimed in claim 1 where R³ is —P(O)(OH)—O—P(O)(OH)—O—P(O)(OH)₂.

6. The compound of formula (I) as claimed in claim 1.

7. The compound of formula (I) as claimed in claim 1 which is a sodium, potassium, or ammonium salt.

8. The compound of formula (I) as claimed in claim 7 which is a monosodium, disodium, trisodium, monoammonium, diammonium or triammonium salt.

9. A process for preparing a compound of formula (I), a physiologically cleavable precursor or a salt thereof as claimed in claim 1 comprising:
i) providing a compound of formula (V) or a salt thereof: 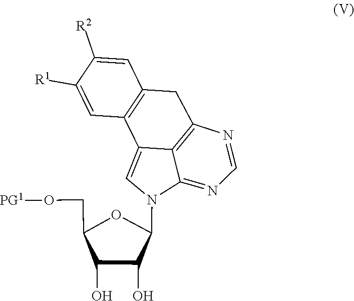
where R¹ is selected from hydro and R² is selected from cyano, or R¹ and R² together with the atoms to which they are attached form a 6-membered carboaromatic ring, and PG¹ is a suitable protecting group;
ii) immobilising the compound of formula (V) or a salt thereof by linking one of its secondary alcohol groups to a suitable support;
iii) capping any free secondary alcohol groups with a suitable protecting group PG²;
iv) removing the protecting group PG¹;
v) reacting the exposed primary alcohol group with a compound of formula (VI): 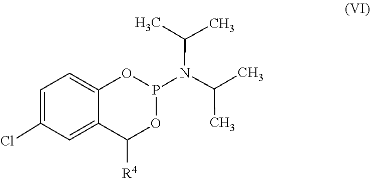
where R⁴ is selected from a hydro group and a C₁₋₃alkyl group;
vi) oxidising the resultant phosphorus (III) compound to a phosphorus (V) compound;
vii) reacting the phosphorus (V) compound with a tetraalkylammonium pyrophosphate to generate a triphosphate;
viii) removing the protecting group PG²;
ix) cleaving the resultant triphosphate from the support to generate a compound of formula (I) or salt thereof; and optionally
x) forming a free acid, physiologically cleavable precursor or different salt of the compound of formula (I).

10. The process for preparing a compound of formula (I), a physiologically cleavable precursor or a salt thereof as claimed in claim 9, where R⁴ is a methyl group.

11. The process for preparing a compound of formula (I), a physiologically cleavable precursor or a salt thereof as claimed in claim 9, where the support is a solid polymer.

12. The process for preparing a compound of formula (I), a physiologically cleavable precursor or a salt thereof as claimed in claim 11, where the support is selected from controlled-porosity glass and polystyrene.

13. The process for preparing a compound of formula (I), a physiologically cleavable precursor or a salt thereof as claimed in claim 12, where the support is controlled-porosity glass.

14. The process for preparing a compound of formula (I), a physiologically cleavable precursor or a salt thereof as claimed in claim 9, where PG¹ is selected from trityl, dimethoxytrityl and trimethoxytrityl.

15. The process for preparing a compound of formula (I), a physiologically cleavable precursor or a salt thereof as claimed in claim 9, where PG² is selected from acetyl, benzoyl, 2,2,2-trichloroethylcarbonyl, paramethoxybenzyl, methyl, tetrahydropyranyl, triethylsilyl, triisopropylsilyl, trimethylsilyl, tert-butyldimethylsilyl and methoxyethyl.

16. The process for preparing a compound of formula (I), a physiologically cleavable precursor or a salt thereof as claimed in claim 9, where PG¹ is dimethoxytrityl and PG² is acetyl.

17. The process for preparing a compound of formula (I), a physiologically cleavable precursor or a salt thereof as claimed in claim 2, where the tetraalkylammonium pyrophosphate is tetrabutylammonium pyrophosphate.

18. A composition for preparing a labelled RNA molecule comprising a compound of formula (I), a physiologically cleavable precursor or a salt thereof as claimed in claim 1 and a natural ribonucleotide.

19. The use of a compound of formula (I), a physiologically cleavable precursor or a salt thereof as claimed in claim 1 to prepare a labelled RNA molecule.

20. The use of a compound of formula (I), a physiologically cleavable precursor or a salt thereof as claimed in claim 19 where R¹ is selected from hydro, R² is selected from cyano and the RNA molecule is labelled with 2CNqA.

21. The use of a compound of formula (I), a physiologically cleavable precursor or a salt thereof as claimed in claim 19 where R¹ and R² together with the atoms to which they are attached form a 6-membered carboaromatic ring and the RNA molecule is labelled with pA.

22. The use of a compound of formula (I), a physiologically cleavable precursor or a salt thereof as claimed in claim 19 where the RNA molecule is selected from mRNA and ribosomal RNA.

23. The use of a compound of formula (I), a physiologically cleavable precursor or a salt thereof as claimed in claim 19 where the RNA molecule is prepared in-cellulo.

24. A process for preparing a labelled RNA molecule in-vitro comprising:
i) providing a DNA template to a composition comprising a compound of formula (I), a physiologically cleavable precursor or a salt thereof as claimed in claim 1 and a natural ribonucleotide;
ii) treating the resultant mixture with an RNA polymerase; optionally
iii) monitoring the labelled RNA molecule using microscopy; and optionally
iv) isolating the labelled RNA molecule.

25. A process for preparing a labelled RNA molecule in-cellulo comprising:
i) providing a compound of formula (I), a physiologically cleavable precursor or a salt thereof as claimed in claim 1 to a eukaryotic or prokaryotic cell;
ii) allowing the organism to spontaneously internalise the compound;
iii) allowing the organism to prepare a labelled RNA molecule; optionally
iv) monitoring the labelled RNA molecule using microscopy; and optionally
v) isolating the labelled RNA molecule.
